(19) United States
(12) Patent Application Publication
(10) Pub. No.: US 2021/0299129 A1
(43) Pub. Date: Sep. 30, 2021

(54) Cardiosafe Antidiabetic Therapy

(71) Applicant: Boehringer Ingelheim International GmbH, Ingelheim am Rhein (DE)

(72) Inventor: Odd-Erik JOHANSEN, Hovik (NO)

(21) Appl. No.: 17/260,235

(22) PCT Filed: Jul. 16, 2019
(86) PCT No.: PCT/EP2019/069131

§ 371 Date: Jan. 14, 2021

(30) Foreign Application Priority Data
Jul. 17, 2018 (EP) 18184034.9
Aug. 3, 2018 (EP) 18187272.2
Sep. 28, 2018 (EP) 18197472.6
Oct. 26, 2018 (EP) 18202843.1
Feb. 13, 2019 (EP) 19157007.6
Feb. 14, 2019 (EP) 19157226.2
May 29, 2019 (EP) 19177388.6

(51) Int. Cl. A61K 31/522; A61P 3/10

(52) U.S. Cl. CPC A61K 31/522; A61P 3/10

(57) ABSTRACT

The present invention relates to methods of using DPP-4 inhibitors, optionally in combination with one or more other active agents, for cardio-safe antidiabetic therapy.

### FIELD OF THE INVENTION

The present invention relates to a certain DPP-4 inhibitor, preferably linagliptin (optionally in combination with one or more other active agents) for use in cardiovascular-safe antidiabetic treatment (especially over long term) of diabetes (preferably type 2 diabetes) patients (especially with early type 2 diabetes), including in (human) patients at increased cardiovascular risk or established complications.

### BACKGROUND OF THE INVENTION

Cardiovascular disease (CVD) is a well-recognized complication of type 2 diabetes mellitus (T2DM) and there is a clinical need for glucose-lowering therapies that do not further increase CV risk in this population.

People who have type 2 diabetes are at an increased risk of cardiovascular disease and, despite recent advancements in treatment options, cardiovascular disease remains the leading cause of death for this population.

It remains unknown how specific agents compare with respect to long-term cardiovascular (CV) effect as only few, long-term head-to-head trials have compared the effects of different diabetes drugs on CV outcomes or CV surrogates, and most have been of relatively short duration with insufficient statistical power.

Furthermore, although, since 2007, new glucose-lowering agents are required by the US Food and Drug Administration (FDA) to demonstrate CV safety before or after regulatory approval, most of these CV outcome trials are conducted in a placebo-controlled setting with no active comparators. Hence, they do not allow an assessment of comparative effectiveness.

Current practice guidelines recommend metformin as first-line therapy in the treatment of individuals with T2DM and provide several treatment options as second-line therapy if glycemic control is not achieved within 3 months of metformin initiation. Both sulfonylureas (SUs) and DPP-4 inhibitors are possible second-line choices (or possible first-line choices in case that metformin is not appropriate or not eligible). SUs were the first available oral glucose-lowering drugs, and have been in clinical use since the late 1950s. The low cost and familiarity of SU in clinical practice are possible factors underlying their continued use as first-line or add-on therapy in T2DM. However, the cardiovascular safety of widely used sulfonylureas remains uncertain.

Glimepiride is a second-generation sulfonylurea which has certain benefits over other SUs and is frequently recommended as a preferred second-line therapy.

Comparison of glimepiride with other first- or second-line treatments, such as a DPP-4 inhibitor particularly linagliptin, for T2DM would be of great interest and relevant to current clinical practice.

Accordingly, there is an aim to investigate the long-term impact on cardiovascular (CV) morbidity and mortality of treatment with linagliptin (5 mg) in comparison with glimepiride (1 to 4 mg) in patients with type 2 diabetes (T2DM) at elevated or high CV risk and receiving usual care, as well as to prove cardiovascular safety of linagliptin versus glimepiride in such patients.

Cardiovascular outcome trials (CVOTs) were reported for three DPP-4 inhibitors approved for clinical use in the European Union: saxagliptin, alogliptin and sitagliptin, each compared to placebo. Safety was uniformly demonstrated across the class for atherosclerotic CV outcomes, with a neutral effect on major adverse CV event (MACE) outcomes for all three agents. However, the safety of the class for heart failure risk was uncertain, with a significant increase of 27% with saxagliptin, which has added to concerns that some antidiabetic agents that stimulate insulin signalling might increase heart failure risk. Non-insulin-related mechanisms have been proposed to additionally contribute to heart failure risk with some DPP-4 inhibitors.

Moreover, long-term renal function is of particular clinical relevance for treatment with DPP-4 inhibitors, which are with the exception of linagliptin renally excreted, necessitating dose adjustment with declining renal function; however, the emerging renal evidence from DPP-4 inhibitor CVOTs to date is incomplete and not consistent across the class. A limitation of the previous CVOTs using saxagliptin, alogliptin and sitagliptin has been that only a minority of patients in the study cohorts had reduced renal function at baseline (estimated glomerular filtration rate (eGFR) <60 ml/min/1.73m2). Even fewer patients had severely reduced renal function (eGFR <30 ml/min/1.73m2) or macroalbuminuria. Indeed, although an estimated ˜50% of patients with T2D are affected by CKD, no previous CVOT has been designed to include this population, and in some cases patients with reduced renal function have been actively excluded. Therefore, despite CKD is one of the most prevalent comorbidities of T2D, there is only scare information available on the long-term clinical safety profile of DPP-4 inhibitors in this important, but under-investigated and clinically challenging patient population with renal burden and at high cardio-renal risk.

Analyses of safety outcomes stratified by baseline renal risk have underscored the increased morbidity experienced by patients with CKD in addition to T2D. Heart failure risk may be of particular concern in these patients, owing to coincident morbidity between heart failure and CKD that is driven by diverse cardio-renal interactions—including haemodynamic and neurohormonal mechanisms. Accordingly, impaired renal function in patients with T2D is a major predictor of excess mortality and adverse outcomes, including CV death and other CV events, and T2D is the most common cause of end-stage renal disease. As renal function naturally declines with age, and diabetes is a life-long disease, even patients without overt CKD are at risk of future development of the comorbidity, and screening for the presence of CKD in patients with T2D is widely recommended.

Therefore, there is need for clinical investigations on the long-term (cardiovascular and renal) safety profile of a DPP-4 inhibitor across a broad range of type 2 diabetes patients, including patients with early type 2 diabetes at elevated CV risk (such as e.g. compared to active comparator) as well as including hitherto under-represented patients at high risk for CV or heart disease and/or chronic kidney disease (CKD), such as with established CV and/or kidney disease (such as e.g. compared to placebo/standard of care).

### SUMMARY OF THE INVENTION

Within the scope of the present invention it has now been found that the certain DPP-4 inhibitor, preferably linagliptin, optionally in combination with one or more other active agents as defined herein (e.g. as monotherapy or as add-on therapy), has properties or effects, which make it useful for the purpose of this invention and/or for fulfilling one or more of the needs or aims mentioned herein.

Linagliptin (5 mg once daily, as monotherapy or as add-on therapy) shows long-term clinical cardiovascular safety as well as certain benefits (e.g. treatment sustainability) in a Cardiovascular Safety Trial (assessing cardiovascular safety compared to glimepiride in patients with type 2 diabetes at increased or high cardiovascular risk or established cardiovascular disease).

The trial has included adults at elevated CV risk or established CV disease and having early type 2 diabetes, such as with a median disease duration of 6.2 years, who either received no treatment at all, or received 1-2 glucose lowering agents (e.g. metformin).

These subjects reflect patients that doctors typically see in their daily clinical practice.

This trial has assessed linagliptin safety over the longest period ever studied in a DPP-4 inhibitor cardiovascular outcome trial, with a median duration and follow-up of more than 6 years.

The Cardiovascular Safety Trial has evaluated CV safety and the long term-term impact on CV morbidity and mortality of treatment with linagliptin (5 mg once daily) versus glimepiride (1 to 4 mg) - each as monotherapy or as add-on therapy- in subjects with type 2 diabetes at increased or high CV risk and receiving usual care (such as added to stable background glucose-lowering medication and cardiovascular standard of care).

Usual care includes both glucose lowering agents (including metformin and/or alpha-glucosidase inhibitors) and cardiovascular drugs (including antihypertensive and lipid lowering agents).

The patients of this Cardiovascular Safety Trial (assessing cardiovascular safety of linagliptin versus glimepiride in patients with type 2 diabetes at increased or high cardiovascular risk or established cardiovascular disease) have been treated with 5 mg linagliptin once daily for a median duration of 5.86 years and observed for a median duration of 6.25 years.

Together with another (placebo-controlled) Cardiovascular (Safety) and Renal Outcomes Trial (assessing cardiovascular safety and kidney/renal microvascular outcome in patients with type 2 diabetes at high or very high risk for heart and/or kidney disease), the present (active comparator) Cardiovascular Safety Trial demonstrates linagliptin's long-term overall safety profile in a broad range of adults with type 2 diabetes, including patients at increased/elevated or high cardiovascular risk (certain risk factors) or established complications (e.g. atherosclerotic cardiovascular disease) as well as patients at high or very high (vascular, e.g. cardiorenal) risk especially for heart and/or kidney disease (e.g. at high or very high risk of cardiovascular and/or renal complications or events).

### BRIEF DESCRIPTION OF THE DRAWINGS

FIG. 1 (Time to first occurrence of 3P-MACE in the Cardiovascular Safety Trial) shows time to first occurrence of three point (3P) MACE (3P-MACE, major adverse cardiac event defined as a cardiovascular death or a nonfatal myocardial infarction (MI) or a nonfatal stroke) in this Cardiovascular Safety Trial.

FIG. 2 (Time to first occurrence of 3P-MACE in the Cardiovascular and Renal Microvascular Outcome Trial) shows time to first occurrence of three point (3P) MACE (3P-MACE, major adverse cardiac event defined as a cardiovascular death or a nonfatal myocardial infarction (MI) or a nonfatal stroke) in this Cardiovascular (Safety) and Renal (Microvascular) Outcome Trial.

### DETAILED DESCRIPTION OF THE INVENTION

In more detail, the following findings have been made:

Cardiovascular Safety Trial

The effect of linagliptin on cardiovascular risk in adult patients with early type 2 diabetes mellitus and increased CV risk or established complications was evaluated in this Cardiovascular Safety Trial, a multi-center, multi-national, randomized, double-blind parallel group trial. The trial compared the risk of experiencing a major adverse cardiovascular event (MACE) between linagliptin and glimepiride when these were added to standard of care (including background therapy with metformin) based on regional standards for HbA1c and CV risk factors. The trial was event driven and patients were followed until at least 631 primary outcome events accrued.

A total of 6033 patients were treated (linagliptin 5 mg=3023; glimepiride 1 mg to 4 mg=3010) and followed for a median of 6.25 years (median time on treatment 5.9 years). Approximately 73% of the study population was Caucasian, 18% was Asian, and 5% was Black. The mean age was 64 years and 60% were male.

At baseline, disease characteristics were balanced. The mean HbA1c was 7.15% and participants had a mean duration of type 2 diabetes mellitus of approximately 7.6 years, further 20% were current smokers. The trial population included 2030 (34%) patients ≥70 years of age, 2089 (35%) patients with cardiovascular disease, and 1130 (19%) patients with renal impairment with an eGFR <60mL/min/1.73m2 at baseline. Overall, the previous use of diabetes medications was balanced across treatment groups (metformin 83% continued as background therapy, sulfonylurea 28% discontinued prior to randomization), patients on insulin were excluded. The use of medications to reduce cardiovascular risk was also balanced (aspirin 47%, statins 65%, ACE inhibitors or ARBs 74%, beta blockers 39%, and calcium channel blockers 30%).

The primary endpoint in this Cardiovascular Safety Trial was the time to first occurrence of three point (3P) MACE. A major adverse cardiac event was defined as a cardiovascular death or a nonfatal myocardial infarction (MI) or a nonfatal stroke. The statistical analysis plan tested for non-inferiority for the occurrence of 3P MACE. The key secondary endpoint evaluated the composite of treatment sustainability defined as the proportion of patients on study treatment, that maintain glycemic control (HbA1c ≤7.0%) without need for additional antidiabetic drug therapy (rescue medication), without any moderate (symptomatic with glucose value <70mg/dL) or severe (requiring assistance) hypoglycemic episodes, and without >2% weight gain from baseline.

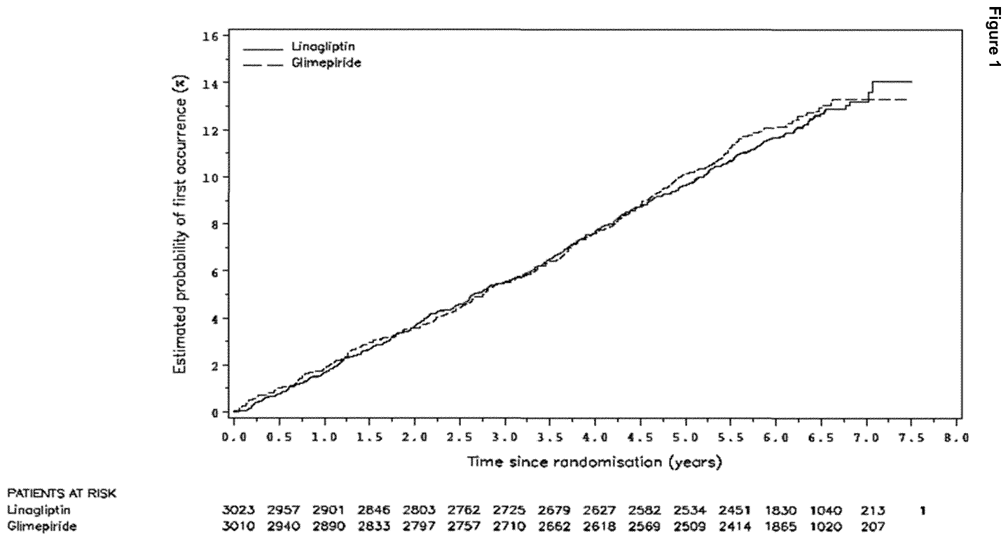

The results of the primary endpoint of this Cardiovascular Safety Trial are shown in Table 1 and FIG. 1. Linagliptin, when added to standard of care, did not increase the risk of major adverse cardiovascular events as compared to glimepiride. The incidence of 3P MACE was similar in both treatment arms; linagliptin (20.7 MACE per 1000 patient years) and glimepiride (21.2 MACE per 1000 patient years). The estimated hazard ratio of MACE associated with linagliptin relative to glimepiride was 0.98 (95% CI; 0.84, 1.14). The upper bound of this confidence interval (CI) 1.14, excluded a pre-defined risk margin larger than 1.3. Results were consistent for patients treated with or without metformin.

TABLE 1
Major Adverse Cardiovascular Events (MACE) and Additional Adjudicated Cardiovascular
and Mortality Endpoints by Treatment Group in the Cardiovascular Safety Trial

| | Linagliptin 5 mg | | Glimepiride (1 mg to 4 mg) | | |
|---|---|---|---|---|---|
| | n = 3023 | | n = 3010 | | |
| | Number of | Incidence | Number | of Incidence | Hazard |
| | Subjects | Rate per | Subjects | Rate per | Ratio |
| | (%) | 1000 PY* | (%) | 1000 PY* | (95% CI) |
| Primary CV composite | 356 (11.8) | 20.7 | 362 (12.0) | 21.2 | 0.98 (0.84, |
| (Cardiovascular death, | | | | | 1.14)** |
| non-fatal MI, non-fatal | | | | | |
| stroke) | | | | | |
| Secondary CV | 398 (13.2) | 23.4 | 401 (13.3) | 23.7 | 0.99 (0.86, |
| composite | | | | | 1.14) |
| (Cardiovascular death, | | | | | |
| non-fatal MI, non-fatal | | | | | |
| stroke and | | | | | |
| hospitalization for | | | | | |
| unstable angina) | | | | | |

*PY = patient years
**Test on non-inferiority to demonstrate that the upper bound of the 95% CI for the hazard ratio is less than 1.3

Vital status was obtained for 99.3% of subjects in the trial. A total of 644 deaths were recorded during the Cardiovascular Safety Trial (Table 2). Of these deaths, 52% were adjudicated as cardiovascular deaths. The risk of deaths from all cause was not statistically different between the treatment groups (HR: 0.91; 95% CI: 0.78, 1.06).

TABLE 2
Major Additional Adjudicated Cardiovascular and Mortality Endpoints by Treatment
Group in the Cardiovascular Safety Trial

| | Linagliptin 5 mg | | Glimepiride (1 mg to 4 mg) | | |
|---|---|---|---|---|---|
| | n = 3023 | | n = 3010 | | |
| | Number of | Incidence | Number of | Incidence | Hazard |
| | Subjects | Rate per | Subjects | Rate per | Ratio |
| | (%) | 1000 PY* | (%) | 1000 PY* | (95% CI) |
| All-cause mortality | 308 (10.2) | 16.8 | 336 (11.2) | 18.4 | 0.91 (0.78, 1.06) |
| CV death | 169 (5.6) | 9.2 | 168 (5.6) | 9.2 | 1.00 (0.81, 1.24) |
| Hospitalization for | 112 (3.7) | 6.4 | 92 (3.1) | 5.3 | 1.21 (0.92, 1.59) |
| heart failure (HHF) | | | | | |
| HHF or CV death | 236 (7.8) | 13.4 | 234 (7.8) | 13.4 | 1.00 (0.84, 1.20) |

The results of the secondary endpoint of the Cardiovascular Safety Trial are shown in Table 3. Following initial titration period (16 weeks), a significantly higher number of patients on treatment with linagliptin achieved sustained glycemic control (HbA1c≤7.0%) without moderate or severe hypoglycemia or substantial weight gain >2% from baseline compared to patients on treatment with glimepiride without a need for additional antidiabetic drug therapy.

TABLE 3
Key secondary composite* by treatment group in the
Cardiovascular Safety Trial

| | Linagliptin | Glimepiride | |
|---|---|---|---|
| | 5 mg | (1 mg to 4 mg) | |
| | n = 3014 | n = 3000 | |
| | Number of | Number of | Odds |
| | Subjects | Subjects | Ratio*** |
| | (%) | (%) | (95% CI) |
| Secondary composite * | 481 (16.0) | 305 (10.2) | 1.68 |
| | | | (1.43, 1.96) |
| Patients with | | | |
| maintained glycemic control | 648 (21.5) | 554 (18.5) | |
| (HbA1c ≤7.0%) | | | |
| any moderate/severe | 163 (5.6) | 791 (27.5) | |
| hypoglycemic episodes** | | | |
| ≤2% weight gain | 775 (25.7) | 699 (23.3) | |

*Secondary composite defined as the composite of treatment sustainability defined as the proportion of patients on study treatment at at final visit, that maintain glycemic control (HbA1c ≤7.0%) from end of stabilisation period (end of titration of study drug after 16 weeks) to final visit on treatment without need for additional antidiabetic medication (rescue) and patients without any moderate/severe hypoglycemic episodes and without >2% weight gain.
**Moderate/severe hypoglycemic episodes defined as:
Moderate hypoglycemic episode: documented symptomatic hypoglycemia with plasma glucose concentration ≤70 mg/dL. Event accompanied by typical symptoms of hypoglycemia but no need for external assistance
Severe hypoglycemic episode: documented hypoglycemic episode requiring assistance of another person to actively administer carbohydrate, glucagon or other resuscitative actions need for external assistance
***Odds ratio and confidence interval based on logistic regression with factor for treatment.

For the entire treatment period (median time on treatment 5.9 years) the rate of moderate or severe hypoglycemia was 6.5% on linagliptin versus 30.9% on glimepiride treatment, severe hypoglycemia (requiring assistance) occurred in 0.3% on linagliptin versus 2.2% on glimepiride treatment.

Accordingly:

The present invention relates to linagliptin, optionally in combination with one or more other active agents, for use in the treatment of diabetic (preferably type 2 diabetes) patients wherein linagliptin effects the treatment without increasing the risk of 3 point major adverse cardiovascular events (3P-MACE) compared to glimepiride, wherein the 3 point major adverse cardiovascular events (3P-MACE) include cardiovascular death, nonfatal myocardial infarction (Ml) and/or nonfatal stroke.

The present invention relates to linagliptin, optionally in combination with one or more other active agents, for use in the treatment of diabetic (preferably type 2 diabetes) patients wherein linagliptin effects the treatment more sustainedly compared with glimepiride, such as characterized in that sustainability responder rates are significantly higher for combined endpoints of patients on trial medication at trial end, with HbA1c≤57.0%, without rescue medication, without >2% weight gain, and with or without moderate/severe hypoglycaemic episodes.

The present invention relates to linagliptin, optionally in combination with one or more other active agents, for use in the treatment of diabetic (preferably type 2 diabetes) patients, wherein linagliptin effects the treatment without increasing risk of deaths from all cause compared to glimepiride.

The present invention relates to linagliptin, optionally in combination with one or more other active agents, for use in the treatment of diabetic (preferably type 2 diabetes) patients wherein linagliptin effects the treatment without increasing the risk of 4 point major adverse cardiovascular events (4P-MACE) compared to glimepiride, wherein the 4 point major adverse cardiovascular events (4P-MACE) include cardiovascular death, nonfatal myocardial infarction (Ml), nonfatal stroke and/or hospitalization for unstable angina pectoris.

The present invention relates to linagliptin, optionally in combination with one or more other active agents, for use in the treatment of a diabetic (preferably type 2 diabetes) patient, wherein linagliptin effects (e.g. at 6.25 years) the treatment as follows:

i) without increasing the risk of (one or more) three point major adverse cardiovascular events (3P-MACE) compared to a patient treated with glimepiride, wherein the one or more three point major adverse cardiovascular events (3P-MACE) are selected from the group consisting of cardiovascular death, nonfatal myocardial infarction (Ml) and nonfatal stroke,

ii) without increasing the risk of (one or more) four point major adverse cardiovascular events (4P-MACE) compared to a patient treated with glimepiride, wherein the one or more three point major adverse cardiovascular events (3P-MACE) are selected from the group consisting of cardiovascular death, nonfatal myocardial infarction (Ml), nonfatal stroke and hospitalization for unstable angina pectoris,

iii) without increasing the risk of deaths from all cause compared to a patient treated with glimepiride,

iv) with resulting in a significantly higher number of patients achieving sustained glycemic control (HbA1c≤7%) without moderate or severe hypoglycemia and/or substantial weight gain >2% from baseline compared to a patient treated with glimepiride, without a need for additional antidiabetic drug therapy, and/or

v) without increasing the risk of CV deaths or hospitalization for heart failure compared to a patient treated with glimepiride.

Patients according to the present invention include patients with T2DM and insufficient glycaemic control, either treatment naïve or despite mono- or dual therapy with metformin and/or an alpha-glucosidase inhibitor or despite a sulphonylurea/glinide in mono- or dual therapy with metformin or an alpha-glucosidase inhibitor (such as HbA1c 6.5 to ≤8.5% if treatment naïve or mono-/dual therapy with metformin and/or an alpha-glucosidase inhibitor; 6.5 to ≤7.5% if treatment with sulphonylurea/glinide in mono- or dual therapy with metformin or an alpha-glucosidase inhibitor).

Patients (especially with T2DM) according to the present invention, who are at increased or high cardiovascular risk or established complications, may have one or more of the following:
a pre-existing CV disease (e.g. selected from: myocardial infarction, coronary artery disease, percutaneous coronary intervention, coronary artery by-pass grafting, ischemic or hemorrhagic stroke, congestive heart failure, peripheral occlusive arterial disease),
a vascular related end-organ damage (e.g. selected from: (moderately) impaired renal function, (micro- or macro)albuminuria, retinopathy),
age >=70 years, and/or
two or more CV risk factors (e.g. selected from: hypertension, smoking, dyslipidemia, duration of T2DM >10 years).

In an embodiment, the diabetic patient has one or more of the following A), B), C) and D):

A) previous or existing vascular disease, such as selected from myocardial infarction, coronary artery disease, percutaneous coronary intervention, coronary artery by-pass grafting, ischemic or hemorrhagic stroke, congestive heart failure, and peripheral occlusive arterial disease,

B) vascular related diabetes end-organ damage, such as selected from (moderately) impaired renal function, (micro- or macro)albuminuria and retinopathy,

C) elderly age (e.g. >/=70 years), and

D) at least two cardiovascular risk factors selected from
advanced type 2 diabetes mellitus (e.g. >10 years duration),
hypertension,
current daily cigarette smoking,
dyslipidemia.

Duration of treatment with linagliptin (preferably 5 mg per day, administered orally, optionally in combination with one or more other active substances, e.g. such as those described herein) for the purpose of the present invention may be over a lengthy period, such as e.g. at least 1-9 years, preferably at least about 5-7 years. In an embodiment, the median treatment exposure is at least about 5.86 years. In an embodiment, the patients are followed for at least 6.25 years.

Other aspects of the present invention become apparent to the skilled person from the foregoing and following remarks (including the examples and claims).

### DETAILED DESCRIPTION OF THE INVENTION

A particularly preferred DPP-4 inhibitor to be emphasized within the present invention is linagliptin. The term “linagliptin” as employed herein refers to linagliptin or a pharmaceutically acceptable salt thereof, including hydrates and solvates thereof, and amorphous or crystalline forms thereof, preferably linagliptin refers to 1-[(4-methyl-quinazolin-2-yl)methyl]-3-methyl-7-(2-butyn-1-yl)-8-(3-(R)-amino-piperidin-1-yl)-xanthine.

Preferably, linagliptin is administered in an oral daily dose of 5 mg (e.g. 2.5 mg twice daily, or —preferably—5 mg once daily).

Further (independent or dependent) aspects:

Cardiovascular (Safety) and Renal Microvascular Outcome Trial

The effect of linagliptin on cardiovascular risk in adult patients with type 2 diabetes mellitus and with increased or high or very high CV risk evidenced by a history of established macrovascular or renal disease was evaluated in a Cardiovascular and Renal Microvascular Outcome Trial, a multi-center, multi-national, randomized, double-blind parallel group trial. The trial compared the risk of experiencing a major adverse cardiovascular event (MACE) between linagliptin and placebo when these were added to and used concomitantly with standard of care treatments for diabetes and other cardiovascular risk factors. The trial was event driven and patients were followed until at least 611 primary outcome events accrued.

A total of 6979 patients were treated (linagliptin 5 mg=3494; placebo=3485) and followed for a median of 2.2 years (median time on treatment 1.9 years). Approximately 80% of the study population was Caucasian, 9% was Asian, and 6% was Black. The mean age was 66 years and 63% were male.

The mean HbA1c at baseline was 8.0% and participants had a mean duration of type 2 diabetes mellitus of approximately 15 years, further 10% were current smokers. The trial population included 1211 (17.4%) patients ≥75 years of age and 4348 (62.3%) patients with renal impairment. Approximately 19% of the population had eGFR ≥45 to <60 mL/min/1.73 m2, 28% of the population had eGFR ≤30 to <45 mL/min/1.73 m2 and 15% had eGFR <30 mL/min/1.73 m2. Overall, the use of diabetes medications was balanced across treatment groups (metformin 54%, sulfonylurea 32%, and insulin 57%). The use of medications to reduce cardiovascular risk was also balanced (aspirin 62%, statins 72%, ACE inhibitors or ARBs 81%, beta blockers 60%, and calcium channel blockers 41%).

The primary endpoint in the Cardiovascular and Renal Microvascular Outcome Trial was the time to first occurrence of three point (3P) MACE. A major adverse cardiac event was defined as a cardiovascular death or a nonfatal myocardial infarction (Ml) or a nonfatal stroke. The statistical analysis plan tested for non-inferiority for the occurrence of (3P) MACE. The secondary endpoint was a renal composite, defined as renal death or sustained end stage renal disease or sustained decrease of 40% or more in eGFR.

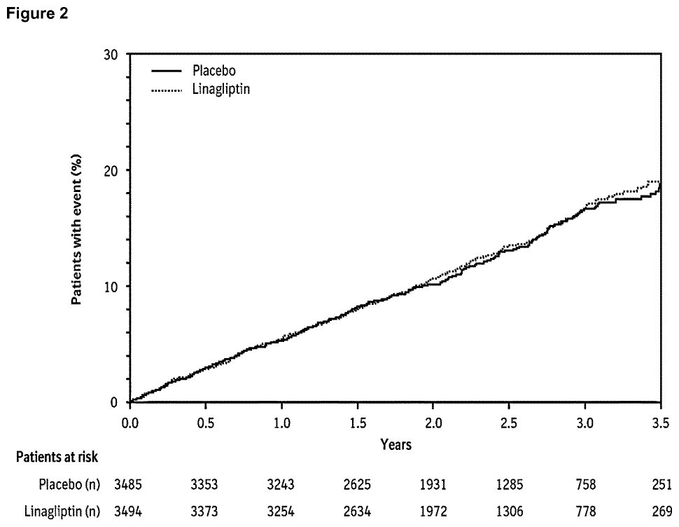

The results of the primary endpoint of the Cardiovascular and Renal Microvascular Outcome Trial are shown in the following Table 4 and FIG. 2. The incidence of (3P) MACE was similar in both treatment arms; placebo (56.3 MACE per 1000 patient years) and linagliptin (57.7 MACE per 1000 patient years). The estimated hazard ratio of MACE associated with linagliptin relative to placebo was 1.02 (95% CI; 0.89, 1.17). The upper bound of this confidence interval 1.17, excluded a pre-defined risk margin larger than 1.3.

TABLE 4
Major Adverse Cardiovascular Events (MACE) by Treatment Group
in the Cardiovascular and Renal Microvascular Outcome Trial

| | TRADJENTA 5 mg | | Placebo | | |
|---|---|---|---|---|---|
| | n = 3494 | | n = 3485 | | Hazard |
| | Number of | Incidence | Number of | Incidence | Ratio |
| | Subjects | Rate per | Subjects | Rate per | (95% |
| | (%) | 1000 PY* | (%) | 1000 PY* | CI) |
| Composite | 434 (12.4) | 57.7 | 420 (12.1) | 56.3 | 1.02 |
| of first event | | | | | (0.89, |
| of CV death, | | | | | 1.17) |
| non-fatal MI, | | | | | |
| or non-fatal stroke | | | | | |
| (MACE) | | | | | |

*PY = patient years

In the Cardiovascular and Renal Microvascular Outcome Trial, there was no increase in the risk of hospitalization for heart failure, which was an additional adjudicated event. The estimated hazard ratio of hospitalization for heart failure associated with linagliptin relative to placebo was 0.90 (95% CI; 0.74, 1.08). In the trial 209 (6.0%) patients treated with linagliptin and 226 (6.5%) patients treated with placebo were hospitalized for heart failure.

Vital status was obtained for 99.7% of subjects in the trial. A total of 740 deaths were recorded during the Cardiovascular and Renal Microvascular Outcome Trial (Table 5). Of these deaths,70% were adjudicated as cardiovascular deaths. The risk of deaths from all cause was not statistically different between the treatment groups (HR: 0.98; 95% CI: 0.84, 1.13).

TABLE 5
Mortality by Treatment Group in the Cardiovascular and Renal
Microvascular Outcome Trial

| | TRADJENTA 5 mg | | Placebo | | |
|---|---|---|---|---|---|
| | n = 3494 | | n = 3485 | | |
| | Number of | Incidence | Number of | Incidence | Hazard |
| | Subjects | Rate per | Subjects | Rate per | Ratio |
| | (%) | 1000 PY* | (%) | 1000 PY* | (95% CI) |
| All-cause | 367 (10.5%) | 46.9 | 373 (10.7%) | 48.0 | 0.98 |
| mortality | | | | | (0.84, |
| | | | | | 1.13) |
| CV death | 255 (7.3%) | 32.6 | 264 (7.6%) | 34.0 | 0.96 |
| | | | | | (0.81, |
| | | | | | 1.14) |

The incidence of the renal composite was similar in both treatment arms (Table 6); placebo (46.6 renal composite per 1000 patient years) and linagliptin (48.9 renal composite per 1000 patient years). The estimated hazard ratio of the renal composite associated with linagliptin relative to placebo was 1.04 (95% CI; 0.89, 1.22).

TABLE 6
Renal outcome events by Treatment Group in the Cardiovascular and
Renal Microvascular Outcome Trial

| | Linagliptin 5 mg | | Placebo | | |
|---|---|---|---|---|---|
| | n = 3494 | | n = 3485 | | |
| | Number of | Incidence | Number of | Incidence | Hazard |
| | Subjects | Rate per | Subjects | Rate per | Ratio |
| | (%) | 1000 PY* | (%) | 1000 PY* | (95% CI) |
| Secondary renal | 327 (9.4) | 48.9 | 306 (8.8) | 46.6 | 1.04 |
| composite | | | | | (0.89, |
| (renal death, | | | | | 1.22) |
| ESRD, 40% | | | | | |
| sustained | | | | | |
| decrease | | | | | |
| in eGFR) | | | | | |

*PY = patient years

In analyses for albuminuria progression (change from normoalbuminuria to micro- or macroalbuminuria, or from microalbuminuria to macroalbuminuria) a hazard ratio of 0.86 (95% Cl 0.78, 0.95) was observed for linagliptin versus placebo.

The microvascular endpoint was defined as the composite of renal death, sustained ESRD, sustained decrease of ≥50% in eGFR, albuminuria progression, use of retinal photocoagulation or intravitreal injections of an anti-VEGF therapy for diabetic retinopathy or vitreous hemorrhage or diabetes-related-blindness. The estimated hazard ratio for time to first occurrence for the composite microvascular endpoint was 0.86 (95% CI 0.78, 0.95) for linagliptin versus placebo; mainly driven by albuminuria progression.

Further, in more detail with regard to the Cardiovascular and Renal Microvascular Outcome Trial:

Around three-quarters of patients in the Cardiovascular and Renal Microvascular Outcome Trial had prevalent CKD at baseline, defined as reduced renal function (eGFR <60 mL/min/1.73 m2) and/or macroalbuminuria (urinary albumin-to-creatinine ratio >300 mg/g).

KDIGO categorises renal prognosis (for adverse kidney events) according to low, moderate, high and very high risk, based on a combination of albuminuria and renal risk. According to this internationally agreed standard, 44% of patients in the Cardiovascular and Renal Microvascular Outcome Trial were at very high risk at baseline and a further 27% of patients were at high risk, with only 7% at low risk.

A limitation of dipeptidyl peptidase-4 (DPP-4) inhibitor cardiovascular outcomes trials (CVOTs) prior to the Cardiovascular and Renal Microvascular Outcome Trial is that only a minority of patients in the study cohorts had reduced renal function at baseline (estimated glomerular filtration rate (eGFR) <60 ml/min/1.73m2). Even fewer patients had severely reduced renal function (eGFR <30 ml/min/1.73m2) or macroalbuminuria (urinary albumin-to-creatinine ratio >300 mg/g). By contrast, 62% and 15% of patients in the Cardiovascular and Renal Microvascular Outcome Trial had reduced or severely reduced renal function at baseline, and the prevalence of macroalbuminuria was 39%, which compares with 10% of patients with macroalbuminuria at baseline in the saxagliptin CVOT. Macroalbuminuria prevalence for the sitagliptin CVOT was based on a limited number of patients for which data were available; prevalence of macroalbuminuria was not reported for the alogliptin CVOT.

The heart and kidneys are intricately linked by diverse interactions that drive a coincident morbidity between heart failure and chronic kidney disease (CKD). Hospitalization for heart failure (HHF) risk is elevated in patients presenting with impaired renal function (as measured by eGFR). However, linagliptin did not affect the risk of HHF, regardless of baseline renal function.

People with type 2 diabetes (T2D) with concomitant chronic kidney disease (CKD) and cardiovascular (CV) disease are at increased risk for recurrent CV events and hypoglycemia. Treatment of these individuals is clinically challenging, where the evidence-base for safety and efficacy of glucose lowering drugs is scarce, in particular in GFR categories G3b (eGFR 30-44 ml/min/1.73m2), G4 (eGFR <30) and G5 (eGFR <15). We analyzed baseline characteristics and effects on CV and kidney outcomes with the DPP-4 inhibitor linagliptin (LINA) vs. placebo (PBO), across GFR categories in the Cardiovascular and Renal Microvascular Outcome Trial. People with T2D and either i) UACR >30 mg/g with concomitant CV disease, or ii) eGFR <45 ml/min/1.73m² regardless of UACR, or eGFR ≥45-75mUmin/1.73m² and UACR >200 mg/g, were randomized to LINA 5 mg or placebo (PBO) q.d. in a double-blind fashion. The primary outcome was first occurrence of CV death, non-fatal myocardial infarction, or non-fatal stroke (3P-MACE), with an adjudicated secondary composite outcome of ESKD, renal death, or sustained ≥40% decrease in eGFR from baseline. Other adjudicated outcomes included hospitalized heart failure (hHF) and the 3P-MACE components. Subgroup-effects across GFR categories (G≤2, G3a, G3b and G≥4) were also assessed. Of the 6979 participants, 15.2% were in GFR category GNI, 27.8% G3b, 19.3% G3a, and 37.7% G2 at baseline. Participants in G≥4 (mean±SD eGFR 23.4±4.2 mL/min/1.73m²) or G3b (eGFR 37.2±4.1) as compared with G3a (eGFR 51.4±4.4) and G≤2 (eGFR 81.6±16.7) had more albuminuria, longer T2D duration and were more frequently treated with insulin, but less often with sulfonylureas and metformin. Over a median 2.2 years, LINA did not affect the risk for 3P-MACE (HR.1.02 [95% CI, 0.89, 1.17]), the secondary kidney composite outcome (1.04 [0.89, 1.22]), hHF (0.90 [0.74, 1.08]), or CV mortality (0.96 [0.81, 1.14]).

Progression of albuminuria category (i.e. change from normoalbuminuria to micro-/macroalbuminuria, or change from microalbuminuria to macroalbuminuria), occurred less frequently in the linagliptin (763/2162 [35.3%],) than in the placebo group (819/2129 [38.5%]); HR 0.86 (95% CI 0.78, 0.95), p=0.003.

Incidences were higher by declining kidney function, e.g. the 3P-MACE PBO incidence rate was 2.4 fold higher in G≥4 (9.6 per 100 patient-yrs) relative to G≤2 (4.0 per 100-patient yrs), whereas the kidney composite 9.8 fold (14.7 vs 1.5 per 100 patient-yrs), hHF 4.1 fold (6.2 vs 1.5 per patient-yrs) and CV death 3.0 fold (6.8 vs 2.3 per 100 patient-yrs) higher, respectively. A consistent neutral effect was observed across all GFR categories (interaction p-values: 0.84 [3P-MACE], 0.36 [kidney composite], 0.88 [hHF], 0.23 [CV mortality]).

Progression of albuminuria was significantly reduced with linagliptin versus placebo overall and a consistent beneficial effect was observed across all eGFR categories (interaction p-value: 0.35).

Adverse events (AE) increased with declining kidney function, but the proportion with ≥1 AE, or ≥1 serious AE were balanced between LINA and PBO across the GFR categories. HbA1c was reduced significantly, but without increased risk for hypoglycemia with LINA vs PBO, across all GFR categories.

Among adults with T2DM and high CV and renal risk, the use of linagliptin compared with placebo, each added to usual care, over a median of 2.2 years resulted in a non-inferior risk of a composite CV outcome with no effect on the secondary kidney outcome.

In this patient population at very high risk for hHF and its complications, linagliptin can be used without increasing the risk for hHF.

These findings in a large, international Cardiovascular (Safety) and Renal Microvascular Outcome Trial in patients with T2D and concomitant CV and renal disease support the safety and tolerability of LINA as a T2D therapy that can be used across a broad range of kidney disease, even including clinically challenging patients (with high cardiorenal risk), where the evidence-base for safety and efficacy of glucose lowering drugs is scarce, in particular in of GFR categories G3b (eGFR 30-44 ml/min/1.73m2), G4 (eGFR <30) and G5 (eGFR <15).

In further context of this Cardiovascular (Safety) and Renal Microvascular Outcome Trial, reference may be made to EP 18184034.9, EP 18187272.2, EP 18197472.6, and EP 18202843.1, the disclosures thereof are hereby incorporated by reference and made a part hereof.

Particular Aspects:

X1. Accordingly, in a further aspect (aspect X1) the present invention relates to linagliptin, optionally in combination with one or more other active agents, for use in the treatment of type 2 diabetes, especially over long term, especially wherein said treatment is characterized by cardiovascular and renal safety (such as e.g. disclosed herein), including in at-risk patients (such as e.g. disclosed herein), such as e.g. having or being at risk of atherosclerotic CV disease, heart failure and/or chronic kidney disease.

X2. Linagliptin, optionally in combination with one or more other active agents, for use according to the present invention (such as e.g. aspect X1), wherein the patients include patients with early type 2 diabetes mellitus and increased CV risk or established complications (such as e.g. disclosed herein).

X3. Linagliptin, optionally in combination with one or more other active agents, for use according to according to the present invention (such as e.g. aspect X1), wherein the patients include patients with increased or high CV risk evidenced by a history of established macrovascular and/or renal disease (such as e.g. disclosed herein).

X4. Linagliptin, optionally in combination with one or more other active agents, for use according to the present invention (such as e.g. aspect X1 or X2), wherein linagliptin effects the treatment as disclosed herein such as follows:

i) without increasing the risk of three point major adverse cardiovascular events (3P-MACE) compared to patients treated with glimepiride, wherein the three point major adverse cardiovascular events (3P-MACE) include cardiovascular death, nonfatal myocardial infarction (MI) and/or nonfatal stroke,

ii) with resulting in a significantly higher number of patients achieving sustained glycemic control (HbA1c <7%) without moderate or severe hypoglycemia and/or substantial weight gain >2% from baseline compared to patients treated with glimepiride, without a need for additional antidiabetic drug therapy,

iii) without increasing the risk of deaths from all cause compared to patients treated with glimepiride,

iv) without increasing the risk of four point major adverse cardiovascular events (4P-MACE) compared to patients treated with glimepiride, wherein the four point major adverse cardiovascular events (4P-MACE) include cardiovascular death, nonfatal myocardial infarction (Ml), nonfatal stroke and/or hospitalization for unstable angina pectoris.

X5. Linagliptin for use according to the present invention (such as e.g. aspect X1 or X3), wherein linagliptin effects the treatment as disclosed herein such as follows:

i) without increasing the risk of three point major adverse cardiovascular events (3P-MACE) compared to patients treated with placebo, wherein the three point major adverse cardiovascular events (3P-MACE) include cardiovascular death, nonfatal myocardial infarction (Ml) and/or nonfatal stroke,

ii) without increasing the risk of hospitalization for heart failure compared to patients treated with placebo,

iii) without increasing the risk of key renal outcome events compared to patients treated with placebo, wherein the key renal outcome events include renal death, sustained end stage renal disease (ESRD) and/or sustained decrease of 40% or more in estimated glomerular filtration rate (eGFR),

iv) with preventing, delaying the occurrence or reducing the risk of albuminuria progression compared to patients treated with placebo, wherein the albuminuria progression includes change from normoalbuminuria to micro- or macroalbuminuria and/or change from microalbuminuria to macroalbuminuria, and/or

v) with preventing, delaying the occurrence or reducing the risk of microvascular renal and/or eye complications compared to patients treated with placebo, wherein the microvascular renal and/or eye complications include renal death, sustained ESRD, sustained decrease of ≥50% in eGFR, albuminuria progression, use of retinal photocoagulation, use of intravitreal injections of an anti-VEGF therapy for diabetic retinopathy, vitreous hemorrhage and/or diabetes-related-blindness.

X6. Linagliptin, optionally in combination with one or more other active agents, for use according to the present invention (such as e.g. aspect X1, X2 or X4), wherein the patient is exposed to treatment for at least 5.9 years, and/or followed for at least 6.25 years.

X7. Linagliptin, optionally in combination with one or more other active agents, for use according to the present invention (such as e.g. aspect X1, X3 or X5), wherein the patient is exposed to treatment for at least 1.8 years or at least 1.9 years, and/or followed for at least 2.2 years.

X8. Linagliptin, optionally in combination with one or more other active agents, for use according to the present invention (such as e.g. aspect X1, X2, X4 or X6), wherein the patient has one or more of the following A), B), C) and D):

A) previous or existing vascular disease, such as selected from myocardial infarction, coronary artery disease, percutaneous coronary intervention, coronary artery by-pass grafting, ischemic or hemorrhagic stroke, congestive heart failure, and peripheral occlusive arterial disease,

B) vascular related diabetes end-organ damage, such as selected from (moderately) impaired renal function, (micro- or macro)albuminuria and retinopathy,

C) elderly age (e.g. >/=70 years), and

D) at least two cardiovascular risk factors selected from
advanced type 2 diabetes mellitus (e.g. >10 years duration),
hypertension,
current daily cigarette smoking,
dyslipidemia.

X9. Linagliptin, optionally in combination with one or more other active agents, for use according to the present invention (such as e.g. aspect X1, X3, X5 or X7), wherein the patient has:

(i) albuminuria (micro or macro) (such as e.g. urine albumin creatinine ratio (UACR) ≥30 mg/g creatinine or ≥30 mg/l (milligram albumin per liter of urine) or ≥30 μg/min (microgram albumin per minute) or ≥30 mg/24 h (milligram albumin per 24 hours)) and previous macrovascular disease, such as e.g. defined as one or more of a) to f):
a) previous myocardial infarction,
b) advanced coronary artery disease,
c) high-risk single-vessel coronary artery disease,
d) previous ischemic or haemorrhagic stroke,
e) presence of carotid artery disease,
f) presence of peripheral artery disease;

and/or

(ii) impaired renal function (e.g. with or without CV co-morbidities), such as e.g. defined by:
impaired renal function (e.g. as defined by MDRD formula) with an estimated glomerular filtration rate (eGFR) 15-45 mL/min/1.73 m² with any urine albumin creatinine ratio (UACR), or
impaired renal function (e.g. as defined by MDRD formula) with an estimated glomerular filtration rate (eGFR) ≥45-75 mL/min/1.73 m² with an urine albumin creatinine ratio (UACR) >200 mg/g creatinine or >200 mg/l (milligram albumin per liter of urine) or >200 μg/min (microgram albumin per minute) or >200 mg/24 h (milligram albumin per 24 hours).

### Further Embodiments

In an embodiment, diabetes patients as referred to herein may include patients who have not previously been treated with an antidiabetic drug (drug-naïve patients). Thus, in an embodiment, the treatments described herein may be used in naïve patients. In certain embodiments of the treatments of this invention, the DPP-4 inhibitor (preferably linagliptin) may be used alone or in combination with one or more other antidiabetics in such patients. In another embodiment, diabetes patients within the meaning of this invention may include patients pre-treated with conventional antidiabetic background medication, such as e.g. patients with advanced or late stage type 2 diabetes mellitus (including patients with failure to conventional antidiabetic therapy), such as e.g. patients with inadequate glycemic control on one, two or more conventional oral and/or non-oral antidiabetic drugs as defined herein, such as e.g. patients with insufficient glycemic control despite (mono-)therapy with metformin, a thiazolidinedione (particularly pioglitazone), a sulphonylurea, a glinide, GLP-1 or GLP-1 analogue, insulin or insulin analogue, or an a-glucosidase inhibitor, or despite dual combination therapy with metformin/sulphonylurea, metformin/thiazolidinedione (particularly pioglitazone), sulphonylurea/ a-glucosidase inhibitor, pioglitazone/sulphonylurea, metformin/insulin, pioglitazone/insulin or sulphonylurea/insulin. Thus, in an embodiment, the treatments described herein may be used in patients experienced with therapy, e.g. with conventional oral and/or non-oral antidiabetic mono- or dual or triple combination medication as mentioned herein. In certain embodiments of the therapies of this invention, in such patients the DPP-4 inhibitor (preferably linagliptin) may be used on top of or added on the existing or ongoing conventional oral and/or non-oral antidiabetic mono- or dual or triple combination medication with which such patients are pre-treated or experienced.

For example, a diabetes patient (particularly type 2 diabetes patient, with insufficient glycemic control) as referred to herein may be treatment-naïve or pre-treated with one or more (e.g. one or two) conventional antidiabetic agents selected from metformin, thiazolidinediones (particularly pioglitazone), sulphonylureas, glinides, a-glucosidase inhibitors (e.g. acarbose, voglibose), and insulin or insulin analogues, such as e.g. pre-treated or experienced with:

metformin, α-glucosidase inhibitor, sulphonylurea or glinide monotherapy, or metformin plus α-glucosidase inhibitor, metformin plus sulphonylurea, metformin plus glinide, α-glucosidase inhibitor plus sulphonylurea, or α-glucosidase inhibitor plus glinide dual combination therapy.

In certain embodiments relating to such treatment-naïve patients, the DPP-4 inhibitor (preferably linagliptin) may be used as monotherapy, or as initial combination therapy such as e.g. with metformin, a thiazolidinedione (particularly pioglitazone), a sulphonylurea, a glinide, an α-glucosidase inhibitor (e.g. acarbose, voglibose), GLP-1 or GLP-1 analogue, or insulin or insulin analogue; preferably as monotherapy.

In certain embodiments relating to such patients pre-treated or experienced with one or two conventional antidiabetic agents, the DPP-4 inhibitor (preferably linagliptin) may be used as as add-on combination therapy, i.e. added to an existing or background therapy with the one or two conventional antidiabetics in patients with insufficient glycemic control despite therapy with the one or more conventional antidiabetic agents, such as e.g. as add-on therapy to one or more (e.g. one or two) conventional antidiabetics selected from metformin, thiazolidinediones (particularly pioglitazone), sulphonylureas, glinides, a-glucosidase inhibitors (e.g. acarbose, voglibose), GLP-1 or GLP-1 analogues, and insulin or insulin analogues, such as e.g.:

as add-on therapy to metformin, to a α-glucosidase inhibitor, to a sulphonylurea or to a glinide;

or as add-on therapy to metformin plus α-glucosidase inhibitor, to metformin plus sulphonylurea, to metformin plus glinide, to α-glucosidase inhibitor plus sulphonylurea, or to α-glucosidase inhibitor plus glinide;

or as add-on therapy to an insulin, with or without metformin, a thiazolidinedione (particularly pioglitazone), a sulphonylurea, a glinide or an α-glucosidase inhibitor (e.g. acarbose, voglibose).

A further embodiment of diabetic patients as described herein may relate to patients ineligible for metformin therapy including
patients for whom metformin therapy is contraindicated, e.g. patients having one or more contraindications against metformin therapy according to label, such as for example patients with at least one contraindication selected from:
   renal disease, renal impairment or renal dysfunction (e.g., as specified by product information of locally approved metformin),
   dehydration,
   unstable or acute congestive heart failure,
   acute or chronic metabolic acidosis, and
   hereditary galactose intolerance;

and
patients who suffer from one or more intolerable side effects attributed to metformin, particularly gastrointestinal side effects associated with metformin, such as for example patients suffering from at least one gastrointestinal side effect selected from:
nausea,
vomiting,
diarrhoea,
intestinal gas, and
severe abdominal discomfort.

A further embodiment of diabetes patients as referred to herein may include, without being limited to, those diabetes patients for whom normal metformin therapy is not appropriate, such as e.g. those diabetes patients who need reduced dose metformin therapy due to reduced tolerability, intolerability or contraindication against metformin or due to (mildly) impaired/reduced renal function (including elderly patients, such as e.g. ≥60-65 years).

A further embodiment of diabetes patients may refer to patients having renal disease, renal dysfunction, or insufficiency or impairment of renal function (including mild, moderate and/or severe renal impairment), e.g. as may be suggested (if not otherwise noted) by elevated serum creatinine levels (e.g. serum creatinine levels above the upper limit of normal for their age, e.g. ≥130-150 μmol/l, or ≥1.5 mg/dl (≥136 μmol/l) in men and ≥1.4 mg/dl (≥124 μmol/l) in women) or abnormal creatinine clearance (e.g. glomerular filtration rate (GFR) ≤30-60 ml/min).

In this context, in a further embodiment, mild renal impairment may be e.g. suggested (if not otherwise noted) by a creatinine clearance of 50-80 ml/min (approximately corresponding to serum creatine levels of ≤1.7 mg/dL in men and ≤1.5 mg/dL in women); moderate renal impairment may be e.g. suggested (if not otherwise noted) by a creatinine clearance of 30-50 ml/min (approximately corresponding to serum creatinine levels of >1.7 to ≤3.0 mg/dL in men and >1.5 to ≤2.5 mg/dL in women); and severe renal impairment may be e.g. suggested (if not otherwise noted) by a creatinine clearance of <30 ml/min (approximately corresponding to serum creatinine levels of >3.0 mg/dL in men and >2.5 mg/dL in women). Patients with end-stage renal disease require dialysis (e.g. hemodialysis or peritoneal dialysis).

In another further embodiment, patients with renal disease, renal dysfunction or renal impairment may include patients with chronic renal insufficiency or impairment, which can be stratified (if not otherwise noted) according to glomerular filtration rate (GFR, ml/min/1.73m²) into 5 disease stages: stage 1 characterized by normal GFR ≥90 (optionally plus either persistent albuminuria (e.g. UACR ≥30 mg/g) or known structural or hereditary renal disease); stage 2 characterized by mild reduction of GFR (GFR 60-89) describing mild renal impairment; stage 3 characterized by moderate reduction of GFR (GFR 30-59) describing moderate renal impairment [or in more detail: stage 3a characterized by mild-moderate reduction of GFR (GFR 45-59) describing mild-moderate renal impairment, stage 3b characterized by moderate-severe reduction of GFR (GFR 30-44) describing moderate-severe renal impairment]; stage 4 characterized by severe reduction of GFR (GFR 15-29) describing severe renal impairment; and terminal stage 5 characterized by requiring dialysis or GFR <15 describing established kidney failure (end-stage renal disease, ESRD).

Chronic kidney disease and its stages (CKD 1-5) can be usually characterized or classified accordingly, such as based on the presence of either kidney damage (albuminuria) or impaired estimated glomerular filtration rate (GFR <60 [ml/min/1.73m²], with or without kidney damage).

Albuminuria stages may be for example classified as disclosed herein and/or by urine albumin creatinine ratio (such as usually UACR ≥30 mg/g, in some instances ≥20 μg/min albumin excretion rate), such as e.g. microalbuminuria may be for example classified by UACR 30-300 mg/g (in some instances 20-200 μg/min) or, in another embodiment, by UACR 30-200 mg/g, and/or macroalbuminuria may be for example classified by UACR >300 mg/g (in some instances >200 μg/min), or, in another embodiment, by UACR >200 mg/g. Very high UACR ≥2000 mg/g may be classified as nephrotic.

A further embodiment of diabetic patients may refer to patients with inadequate control of albuminuria despite therapy with an angiotensin-converting enzyme (ACE) inhibitor and/or an angiotensin II receptor blocker (ARB).

A further embodiment of diabetic patients may refer to patients (preferably diabetic patients, particularly type 2 diabetes patients) having micro- (renal-) and/or macro- (cardiovascular-) disease history and/or medications, such as CKD/diabetic nephropathy, renal impairment and/or (micro- or macro)albuminuria, and/or macrovascular disease (e.g. coronary artery disease, peripheral artery disease, cerebrovascular disease, hypertension), and/or microvascular disease (e.g. diabetic nephropathy, neuropathy, retinopathy), and/or on acetylsalicylic acid, antihypertensive and/or lipid lowering medication, such as e.g. on (previous or ongoing) therapy with acetylsalicylic acid, an ACE inhibitor, ARB, beta-blocker, Calcium-antagonist or diuretic, or combination thereof, and/or on (previous or ongoing) therapy with a fibrate, niacin or statin, or combination thereof.

The DPP-4 inhibitor may be administered in combination (e.g. on-top, add-on) with the background medication such as e.g. angiotensin-converting enzyme (ACE) inhibitor or the angiotensin II receptor blocker (ARB), to the patient.

Within this invention it is to be understood that combinations, compositions or combined uses according to this invention may envisage the simultaneous, sequential or separate administration of the active components or ingredients.

In this context, “combination” or “combined” within the meaning of this invention may include, without being limited, fixed and non-fixed (e.g. free) forms (including kits) and uses, such as e.g. the simultaneous, sequential or separate use of the components or ingredients.

The combined administration of this invention may take place by administering the active components or ingredients together, such as e.g. by administering them simultaneously in one single or in two separate formulations or dosage forms. Alternatively, the administration may take place by administering the active components or ingredients sequentially, such as e.g. successively in two separate formulations or dosage forms.

For the combination therapy of this invention the active components or ingredients may be administered separately (which implies that they are formulated separately) or formulated altogether (which implies that they are formulated in the same preparation or in the same dosage form). Hence, the administration of one element of the combination of the present invention may be prior to, concurrent to, or subsequent to the administration of the other element of the combination.

Unless otherwise noted, combination therapy may refer to first line, second line or third line therapy, or initial or add-on combination therapy or replacement therapy.

Unless otherwise noted, monotherapy may refer to first line therapy (e.g. therapy of patients with insufficient glycemic control by diet and exercise alone, such as e.g. drug-naive patients, typically patients early after diagnosis and/or who have not been previously treated with an antidiabetic agent, and/or patients ineligible for metformin therapy such as e.g. patients for whom metformin therapy is contraindicated, such as e.g. due to renal impairment, or inappropriate, such as e.g. due to intolerance).

Unless otherwise noted, add-on combination therapy may refer to second line or third line therapy (e.g. therapy of patients with insufficient glycemic control despite (diet and exercise plus) therapy with one or two conventional antidiabetic agents, typically patients who are pre-treated with one or two antidiabetic agents, such as e.g. patients with such existing antidiabetic background medication).

Unless otherwise noted, initial combination therapy may refer to first line therapy (e.g. therapy of patients with insufficient glycemic control by diet and exercise alone, such as e.g. drug-naive patients, typically patients early after diagnosis and/or who have not been previously treated with an antidiabetic agent).

As different metabolic functional disorders often occur simultaneously, it is quite often indicated to combine a number of different active principles with one another. Thus, depending on the functional disorders diagnosed, improved treatment outcomes may be obtained if a DPP-4 inhibitor is combined with one or more active substances customary for the respective disorders, such as e.g. one or more active substances selected from among the other antidiabetic substances, especially active substances that lower the blood sugar level or the lipid level in the blood, raise the HDL level in the blood, lower blood pressure or are indicated in the treatment of atherosclerosis or obesity.

The DPP-4 inhibitors mentioned above—besides their use in mono-therapy—may also be used in conjunction with one or more other active substances, by means of which improved treatment results can be obtained. Such a combined treatment may be given as a free combination of the substances or in the form of a fixed combination, for example in a tablet or capsule. Pharmaceutical formulations of the combination partner needed for this may either be obtained commercially as pharmaceutical compositions or may be formulated by the skilled man using conventional methods. The active substances which may be obtained commercially as pharmaceutical compositions are described in numerous places in the prior art, for example in the list of drugs that appears annually, the “Rote Liste®” of the federal association of the pharmaceutical industry, or in the annually updated compilation of manufacturers' information on prescription drugs known as the “Physicians' Desk Reference”.

Examples of antidiabetic combination partners are metformin; sulphonylureas such as glibenclamide, tolbutamide, glimepiride, glipizide, gliquidon, glibornuride and gliclazide;

nateglinide; repaglinide; mitiglinide; thiazolidinediones such as rosiglitazone and pioglitazone; alpha-glucosidase blockers such as acarbose, voglibose and miglitol; insulin and insulin analogues such as human insulin, insulin lispro, insulin glusilin, r-DNA-insulinaspart, NPH insulin, insulin detemir, insulin degludec, insulin tregopil, insulin zinc suspension and insulin glargin; amylin and amylin analogues (e.g. pramlintide or davalintide); GLP-1 and GLP-1 analogues such as Exendin-4, e.g. exenatide, exenatide LAR, liraglutide, taspoglutide, lixisenatide (AVE-0010), LY-2428757 (a PEGylated version of GLP-1), dulaglutide (LY-2189265), semaglutide or albiglutide; and/or SGLT2-inhibitors such as e.g. dapagliflozin, sergliflozin (KGT-1251), atigliflozin, canagliflozin, ipragliflozin, luseogliflozin or tofogliflozin.

Metformin is usually given in doses varying from about 500 mg to 2000 mg up to 2500 mg per day using various dosing regimens from about 100 mg to 500 mg or 200 mg to 850 mg (1-3 times a day), or about 300 mg to 1000 mg once or twice a day, or delayed-release metformin in doses of about 100 mg to 1000 mg or preferably 500 mg to 1000 mg once or twice a day or about 500 mg to 2000 mg once a day. Particular dosage strengths may be 250, 500, 625, 750, 850 and 1000 mg of metformin hydrochloride.

A dosage of pioglitazone is usually of about 1-10 mg, 15 mg, 30 mg, or 45 mg once a day. Rosiglitazone is usually given in doses from 4 to 8 mg once (or divided twice) a day (typical dosage strengths are 2, 4 and 8 mg).

Glibenclamide (glyburide) is usually given in doses from 2.5-5 to 20 mg once (or divided twice) a day (typical dosage strengths are 1.25, 2.5 and 5 mg), or micronized glibenclamide in doses from 0.75-3 to 12 mg once (or divided twice) a day (typical dosage strengths are 1.5, 3, 4.5 and 6 mg).

Glipizide is usually given in doses from 2.5 to 10-20 mg once (or up to 40 mg divided twice) a day (typical dosage strengths are 5 and 10 mg), or extended-release glibenclamide in doses from 5 to 10 mg (up to 20 mg) once a day (typical dosage strengths are 2.5, 5 and 10 mg).

Glimepiride is usually given in doses from 1-2 to 4 mg (up to 8 mg) once a day (typical dosage strengths are 1, 2 and 4 mg).

A dual combination of glibenclamide/metformin is usually given in doses from 1.25/250 once daily to 10/1000 mg twice daily. (typical dosage strengths are 1.25/250, 2.5/500 and 5/500 mg).

A dual combination of glipizide/metformin is usually given in doses from 2.5/250 to 10/1000 mg twice daily (typical dosage strengths are 2.5/250, 2.5/500 and 5/500 mg).

A dual combination of glimepiride/metformin is usually given in doses from 1/250 to 4/1000 mg twice daily.

A dual combination of rosiglitazone/glimepiride is usually given in doses from 4/1 once or twice daily to 4/2 mg twice daily (typical dosage strengths are 4/1, 4/2, 4/4, 8/2 and 8/4 mg).

A dual combination of pioglitazone/glimepiride is usually given in doses from 30/2 to 30/4 mg once daily (typical dosage strengths are 30/4 and 45/4 mg).

A dual combination of rosiglitazone/metformin is usually given in doses from 1/500 to 4/1000 mg twice daily (typical dosage strengths are 1/500, 2/500, 4/500, 2/1000 and 4/1000 mg).

A dual combination of pioglitazone/metformin is usually given in doses from 15/500 once or twice daily to 15/850 mg thrice daily (typical dosage strengths are 15/500 and 15/850 mg).

The non-sulphonylurea insulin secretagogue nateglinide is usually given in doses from 60 to 120 mg with meals (up to 360 mg/day, typical dosage strengths are 60 and 120 mg); repaglinide is usually given in doses from 0.5 to 4 mg with meals (up to 16 mg/day, typical dosage strengths are 0.5, 1 and 2 mg). A dual combination of repaglinide/metformin is available in dosage strengths of 1/500 and 2/850 mg.

Acarbose is usually given in doses from 25 to 100 mg with meals. Miglitol is usually given in doses from 25 to 100 mg with meals.

Examples of combination partners that lower the lipid level in the blood are HMG-CoA-reductase inhibitors such as simvastatin, atorvastatin, lovastatin, fluvastatin, pravastatin, pitavastatin and rosuvastatin; fibrates such as bezafibrate, fenofibrate, clofibrate, gemfibrozil, etofibrate and etofyllinclofibrate; nicotinic acid and the derivatives thereof such as acipimox; PPAR-alpha agonists; PPAR-delta agonists; PPAR- alpha/delta agonists; inhibitors of acyl-coenzyme A:cholesterolacyltransferase (ACAT; EC 2.3.1.26) such as avasimibe; cholesterol resorption inhibitors such as ezetimib; substances that bind to bile acid, such as cholestyramine, colestipol and colesevelam; inhibitors of bile acid transport; HDL modulating active substances such as D4F, reverse D4F, LXR modulating active substances and FXR modulating active substances; CETP inhibitors such as torcetrapib, JTT-705 (dalcetrapib) or compound 12 from WO 2007/005572 (anacetrapib); LDL receptor modulators; MTP inhibitors (e.g. lomitapide); and ApoB100 antisense RNA.

A dosage of atorvastatin is usually from 1 mg to 40 mg or 10 mg to 80 mg once a day.

Examples of combination partners that lower blood pressure are beta-blockers such as atenolol, bisoprolol, celiprolol, metoprolol and carvedilol; diuretics such as hydrochlorothiazide, chlortalidon, xipamide, furosemide, piretanide, torasemide, spironolactone, eplerenone, amiloride and triamterene; calcium channel blockers such as amlodipine, nifedipine, nitrendipine, nisoldipine, nicardipine, felodipine, lacidipine, lercanipidine, manidipine, isradipine, nilvadipine, verapamil, gallopamil and diltiazem; ACE inhibitors such as ramipril, lisinopril, cilazapril, quinapril, captopril, enalapril, benazepril, perindopril, fosinopril and trandolapril; as well as angiotensin II receptor blockers (ARBs) such as telmisartan, candesartan, valsartan, losartan, irbesartan, olmesartan, azilsartan and eprosartan.

A dosage of telmisartan is usually from 20 mg to 320 mg or 40 mg to 160 mg per day.

Examples of combination partners which increase the HDL level in the blood are Cholesteryl Ester Transfer Protein (CETP) inhibitors; inhibitors of endothelial lipase; regulators of ABC1; LXRalpha antagonists; LXRbeta agonists; PPAR-delta agonists; LXRalpha/beta regulators, and substances that increase the expression and/or plasma concentration of apolipoprotein A-I.

Examples of combination partners for the treatment of obesity are sibutramine; tetrahydrolipstatin (orlistat); alizyme (cetilistat); dexfenfluramine; axokine; cannabinoid receptor 1 antagonists such as the CB1 antagonist rimonobant; MCH-1 receptor antagonists; MC4 receptor agonists; NPY5 as well as NPY2 antagonists (e.g. velneperit); beta3-AR agonists such as SB-418790 and AD-9677; 5HT2c receptor agonists such as APD 356 (lorcaserin); myostatin inhibitors; Acrp30 and adiponectin; steroyl CoA desaturase (SCD1) inhibitors; fatty acid synthase (FAS) inhibitors; CCK receptor agonists; Ghrelin receptor modulators; Pyy 3-36; orexin receptor antagonists; and tesofensine; as well as the dual combinations bupropion/naltrexone, bupropion/zonisamide, topiramate/phentermine and pramlintide/metreleptin.

Examples of combination partners for the treatment of atherosclerosis are phospholipase A2 inhibitors; inhibitors of tyrosine-kinases (50 mg to 600 mg) such as PDGF-receptor-kinase (cf. EP-A-564409, WO 98/35958, U.S. Pat. No. 5,093,330, WO 2004/005281, and WO 2006/041976); oxLDL antibodies and oxLDL vaccines; apoA-1 Milano; ASA; and VCAM-1 inhibitors.

Further, the certain DPP-4 inhibitor of this invention may be used in combination with a substrate of DPP-4 (particularly with an anti-inflammatory substrate of DPP-4), which may be other than GLP-1, for the purposes according to the present invention, such substrates of DPP-4 include, for example—without being limited to, one or more of the following:

Incretins:

Glucagon-like peptide (GLP)-1

Glucose-dependent insulinotropic peptide (GIP)

Neuroactive:

Substance P

Neuropeptide Y (NPY)

Peptide YY

Energy homeostasis:

GLP-2

Prolactin

Pituitary adenylate cyclase activating peptide (PACAP)

Other Hormones:

PACAP 27

Human chorionic gonadotrophin alpha chain

Growth hormone releasing factor (GHRF)

Luteinizing hormone alpha chain

Insulin-like growth factor (IGF-1)

CCL8/eotaxin

CCL22/macrophage-derived chemokine

CXCL9/interferon-gamma-induced monokine

Chemokines:

CXCL10/interferon-gamma-induced protein-10

CXCL11/interferon-inducible T cell a chemoattractant

CCL3L1/macrophage inflammatory protein lalpha isoform

LD78beta

CXCL12/stromal-derived factor 1 alpha and beta

Other:

Enkephalins, gastrin-releasing peptide, vasostatin-1, peptide histidine methionine, thyrotropin alpha

Further or in addition, the certain DPP-4 inhibitor of this invention may be used in combination with one or more active substances which are indicated in the treatment of nephropathy, such as selected from diuretics, ACE inhibitors and/or ARBs.

Further or in addition, the certain DPP-4 inhibitor of this invention may be used in combination with one or more active substances which are indicated in the treatment or prevention of cardiovascular diseases or events (e.g. major cardiovascular events).

Moreover, optionally in addition, the certain DPP-4 inhibitor of this invention may be used in combination with one or more antiplatelet agents, such as e.g. (low-dose) aspirin (acetylsalicylic acid), a selective COX-2 or nonselective COX-1/COX-2 inhibitor, or a ADP receptor inhibitor, such as a thienopyridine (e.g. clopidogrel or prasugrel), elinogrel or ticagrelor, or a thrombin receptor antagonist such as vorapaxar.

Yet moreover, optionally in addition, the certain DPP-4 inhibitor of this invention may be used in combination with one or more anticoagulant agents, such as e.g. heparin, a coumarin (such as warfarin or phenprocoumon), a pentasaccharide inhibitor of Factor Xa (e.g. fondaparinux), or a direct thrombin inhibitor (such as e.g. dabigatran), or a Faktor Xa inhibitor (such as e.g. rivaroxaban or apixaban or edoxaban or otamixaban).

Still yet moreover, optionally in addition, the certain DPP-4 inhibitor of this invention may be used in combination with one or more agents for the treatment of heart failure (such as e.g. those mentioned in WO 2007/128761).

The present invention is not to be limited in scope by the specific embodiments described herein. Various modifications of the invention in addition to those described herein may become apparent to those skilled in the art from the present disclosure. Such modifications are intended to fall within the scope of the appended claims.

All patent applications cited herein are hereby incorporated by reference in their entireties.

### EXAMPLES

In order that this invention be more fully understood, the herein-given examples are set forth. Further embodiments, features, effects, properties or aspects of the present invention may become apparent from the examples. The examples serve to illustrate, by way of example, the principles of the invention without restricting it.

Cardiovascular Outcome Trial of linagliptin versus glimepriride in type 2 diabetes -assessing long-term cardiovascular impact of linagliptin versus the sulphonylurea glimepiride in subjects with early type 2 diabetes and increased cardiovascular risk or established complications.

Treatment of patients with type 2 diabetes mellitus at increased or high cardiovascular risk

The long-term impact on cardiovascular morbidity and mortality and relevant efficacy parameters (e.g. HbA1c, fasting plasma glucose, treatment sustainability) of treatment with linagliptin in a relevant population of patients with type 2 diabetes mellitus is investigated as follows:

Type 2 diabetes patient with insufficient glycemic control (naïve or currently treated (mono or dual therapy) with e.g. metformin and/or an alpha-glucosidase inibitor (e.g. having HbA1c 6.5-8.5%), or currently treated (mono or dual therapy) with e.g. a sulphonylurea or glinide, with or without metformin or an alpha-glucosidase inhibitor (e.g. having HbA1c 6.5-7.5%)) and increased or high risk of cardiovascular events, e.g. defined as one or more of risk factors A), B), C) and D) indicated below, are randomized* and treated over a lengthy period (e.g. up to 432 weeks) with linagliptin (5 mg, q.d., optionally in combination with one or more other active substances, e.g. such as added to existing antidiabetic background) and compared with patients treated with the sulphonylurea glimepiride (1-4 mg once daily). * Upon randomization, if a patient is on a SU or glinide, this secretagogue therapy is discontinued and replaced with study medication. For patients not on a secretagogue, study medication is added to the existing regimen.

Efficacy Criteria for Evaluation

Primary Endpoint

Time to first occurrence of any of the following adjudicated components of the primary composite endpoint:

CV death (including fatal stroke and fatal MI), non-fatal stroke, or non-fatal MI (excluding silent MI) (also referred to as ‘time to first 3-point Major Adverse Cardiovascular Events [3P-MACE]’).

Key secondary endpoints

1) Time to first occurrence of any of the following adjudicated components of the composite endpoint: CV death (including fatal stroke and fatal MI), non-fatal stroke, non-fatal MI (excluding silent MI), or hospitalisation for unstable angina pectoris (also referred to as ‘time to first 4P-MACE’).

2) Composite endpoint of treatment sustainability: proportion of patients on trial medication at study end, who maintained glycaemic control at Final Visit (HbA1c≤7.0%), without need for rescue medication (between end of titration at Visit 6 and Final Visit), without >2% weight gain (between Visit 6 and Final Visit), and without moderate/severe hypoglycaemic episodes (between Visit 6 and Final Visit).

3) Composite endpoint of treatment sustainability: proportion of patients on trial medication at study end, who maintained glycaemic control at Final Visit (HbA1c≤7.0%), without need for rescue medication (between Visit 6 and Final Visit), and without >2% weight gain (between

Visit 6 and Final Visit).

Secondary endpoints

Secondary CV endpoints:
Occurrence of at least one of the following adjudicated components of CV death (including fatal stroke and fatal MI), non-fatal MI (excluding silent MI), and non-fatal stroke (also referred to as ‘occurrence of 3P-MACE’).
Occurrence of at least one of the following adjudicated components of CV death (including fatal stroke and fatal MI), non-fatal MI (excluding silent MI), non-fatal stroke, and hospitalisation for unstable angina pectoris (also referred to as ‘occurrence of 4P-MACE’).
Occurrence of and time to the first occurrence of any of the following components of the composite endpoint of all adjudicated events: CV death (including fatal stroke and fatal MI), non-fatal MI, non-fatal stroke, hospitalisation for unstable angina pectoris, transient ischaemic attack (TIA), hospitalisation for heart failure, hospitalisation for coronary revascularisation procedures (CABG, PCI).

Secondary diabetes-related endpoints
Change from baseline to Final Visit in the following laboratory parameters: HbA1c, fasting plasma glucose (FPG), total cholesterol, low-density lipoprotein (LDL) cholesterol, high-density lipoprotein (HDL) cholesterol, triglycerides, creatinine, estimated glomerular filtration rate (eGFR, MDRD formula), urinary albumin.
Any transition in albuminuria categories based on urine albumin-to-creatinine ratio (UACR).

Risk factors A), B), C) and D) for cardiovascular events:
A) Previous (cardio)vascular disease (e.g. age 40-85 years):
   myocardial infarction (e.g. >=6 weeks),
   coronary artery disease (e.g. >=50% luminal diameter narrowing of left main coronary artery or in at least two major coronary arteries in angiogram),
   percutaneous coronary intervention (e.g. >=6 weeks),
   coronary artery by-pass grafting (e.g. >=4 years or with recurrent angina following surgery),
   ischemic or hemorrhagic stroke (e.g. >=3 months),
   peripheral occlusive arterial disease (e.g. previous limb bypass surgery or percutaneous transluminal angioplasty; previous limb or foot amputation due to circulatory insufficiency, angiographic or ultrasound detected significant vessel stenosis (>50%) of major limb arteries (common iliac artery, internal iliac artery, external iliac artery, femoral artery, popliteal artery), history of intermittent claudication, with an ankle: arm blood pressure ratio <0.90 on at least one side),
B) Vascular related diabetes end-organ damage (e.g. age 40-85 years):
   impaired renal function (e.g. moderately impaired renal function as defined by MDRD formula, with eGFRF 30-59 mL/min/1.73m2),
   micro- or macroalbuminuria (e.g. microalbuminuria, or random spot urinary albumin:creatinine ratio >/=30 pg/mg),
   retinopathy (e.g. proliferative retinopathy, or retinal neovascularisation or previous retinal laser coagulation therapy),
C) Elderly (e.g. age >/=70 years),
D) At least two of the following cardiovascular risk factors (e.g. age 40-85 years):
   advanced type 2 diabetes mellitus (e.g. >10 years duration),
   hypertension (e.g. systolic blood pressure >140 mmHg or on at least one blood pressure lowering treatment, e.g. <=6 months),
   current daily cigarette smoking,
   (atherogenic) dyslipidemia or high LDL cholesterol blood levels (e.g. LDL cholesterol >/=135 mg/dL) or on at least one treatment for lipid abnormality (e.g. <=6 months).

Results

Summary Conclusions

Trial Patients and Compliance with Trial Protocol:

Disposition

A total of 6077 patients were randomised and 6068 were treated. The data from patients at one site were excluded from the analysis datasets; the TS therefore consisted of 6033 patients. Overall, the frequency of premature discontinuation from the trial was low and balanced between the two groups (linagliptin: 4.1%, glimepiride: 3.8%). Less than 1% of patients were lost to follow-up for vital status (linagliptin: 0.8%, glimepiride: 0.7%). Premature discontinuation from trial medication was comparable between treatment groups (linagliptin: 37.3%, glimepiride: 39.1%), most frequently due to adverse events (AEs; linagliptin: 15.1%, glimepiride 17.4%) or because the patient refused to continue taking the trial medication not due to an AE (linagliptin: 10.2%, glimepiride: 10.9%).

Demographics

The population of the trial was as intended and as per the inclusion criteria, patients had an increased risk of CV events. Demographic characteristics were well balanced across the treatment groups. The main baseline characteristics (mean and standard deviation [SD] or proportion of patients) were the following:
Patients were 64.0 years (9.5 years) old.
60.0% of patients were male and 40.0% were female.
Patients were predominantly White (73.0%) or Asian (17.6%); 17.1% were Hispanic or Latino.
The time since the diagnosis was 7.6 years (6.1 years).
Patients had a BMI of 30.08 kg/m2 (5.1 kg/m2).
Patients had an HbA1c level of 7.15% (0.57%).
Patients had a fasting plasma glucose (FPG) level of 140.0 mg/dL (30.5 mg/dL).
Patients had an eGFR (MDRD formula) of 76.8 mL/min/1.73m2 (19.8 mL/min/1.73m2).
The proportion of patients with UACR <30 mg/g was 74.0%

Exposure

The median time in trial was 6.25 years in both treatment groups and the median exposure to trial medication was 5.86 years in both treatment groups.

Efficacy results

3P-MACE and 4P-MACE (primary and first key secondary endpoints) and components The primary endpoint was met and linagliptin was demonstrated to be non-inferior to glimepiride for the time to 3P-MACE, as the upper bound of the 95.47% Cl was below 1.3 (one-sided p<0.0001). In total, 356 patients (11.8%) were reported with a 3P-MACE event in the linagliptin group and 362 patients (12.0%) in the glimepiride group (HR=0.98; 95.47% Cl 0.84, 1.14). As the upper bound of the 95.47% Cl was above 1.0, the next step in the testing hierarchy, the analysis of the primary endpoint for superiority of linagliptin, was not met (one-sided p=0.3813). The proportion of patients with each event type contributing to the 3PMACE was generally balanced between the treatment groups (CV death: linagliptin 4.3%, glimepiride 4.2%; non-fatal MI: 4.7% vs. 4.6%; non-fatal stroke: 2.8% vs. 3.4%). As the testing hierarchy was not fulfilled, all further analyses were considered exploratory. Consistent with the primary endpoint, no treatment difference was shown for the time to first 4P-MACE (HR=0.99; 95.47% CI 0.86, 1.14; one-sided p=0.4334). There were 398 patients (13.2%) with a 4P-MACE event in the linagliptin group and 401 patients (13.3%) in the glimepiride group. The proportion of patients with each event type contributing to the 4P-MACE was generally balanced between the treatment groups (CV death: linagliptin 4.1%, glimepiride 4.1%; non-fatal MI: 4.4% vs. 4.3%; non-fatal stroke: 2.8% vs. 3.3%; hospitalisation for unstable angina pectoris: 1.9% vs. 1.8%).

The sensitivity analyses for the primary and key secondary endpoints used different analysis sets and censoring approaches and the results were consistent with the main analyses. Likewise, consistent results were generally observed across the subgroups. Further analyses showed no significant treatment differences for all-cause mortality (linagliptin 10.2% of patients, incidence rate per 1000 years at risk 16.8; glimepiride 12.3%, 18.4; hazard ratio 0.91, 95% CI 0.78, 1.06, p=0.2263), CV death (both groups 5.6%, 9.2; HR 1.00, 95% CI 0.81, 1.24, p=0.9863), and non-CV death (linagliptin 4.6% of patients, incidence rate per 1000 years at risk 7.6; glimepiride 5.6%, 9.2; hazard ratio 0.82, 95% Cl 0.66, 1.03, p=0.0839).

In addition, heart failure outcomes were analysed. There was no significant treatment difference for hospitalisation for heart failure alone: linagliptin 3.7% of patients, incidence rate per 1000 years at risk 6.4; glimepiride 3.1%, 5.3; hazard ratio 1.21, 95% CI 0.92, 1.59, p=0.1761, or when analysed combined with CV death (both groups 7.8%, 13.4; HR 1.00, 95% Cl 0.84, 1.20, p=0.9671). Similarly, there was no significant treatment difference for investigator-reported heart failure AEs (linagliptin 5.5%, 9.5; glimepiride 5.2%, 9.0; HR 1.06, 95% CI 0.85, 1.32, p=0.5844).

Treatment sustainability (second and third key secondary endpoints) and components Both second and third key secondary endpoints consistently showed that linagliptin had a higher treatment sustainability compared with glimepiride; there was a higher rate of responders in the linagliptin group compared with the glimepiride group. For the second key secondary endpoint, 16.0% of patients in the linagliptin group and 10.2% in the glimepiride group were responders (OR=1.68; 95.47% CI 1.43, 1.96; p<0.0001). For the third key secondary endpoint, 17.4% of patients in the linagliptin group and 14.1% in the glimepiride group were responders (OR=1.29; 95.47% CI 1.11, 1.48; p=0.0004). The sensitivity and subgroup analyses were generally consistent with the main analyses. Further analyses of the components of the treatment sustainability endpoints showed that the proportion of patients with moderate/severe hypoglycaemic episodes between end of titration at Visit 6 and Final Visit was significantly lower in the linagliptin group compared with glimepiride (linagliptin: 5.6% of patients, glimepiride: 27.5%; odds ratio 0.16; 95% CI 0.13, 0.19; p<0.0001).

In addition, while the glimepiride group showed an initial numerical weight increase, the linagliptin group showed a modest decrease in weight with a significant and sustained difference compared with the glimepiride group over time (adjusted mean difference −1.61 kg; 95% Cl −1.92, −1.29; p<0.0001).

Patients in the linagliptin group had a significantly shorter time to first intake of rescue medication (incidence rate per 1000 years at risk: linagliptin 128.6, glimepiride 115.5; p=0.0035), while the rate of patients who used rescue medication was comparable between both treatment groups (linagliptin: 49.3%, glimepiride: 47.1%). This is in line with a larger initial decrease of HbA1c in the glimepiride group at the end of titration. Over the course of the trial, HbA1c differences between treatment groups narrowed and the slopes crossed, indicating comparable HbA1c control over time in both treatment groups/a more sustained HbA1c lowering effect of linagliptin compared with glimepiride over time (see Table 7 for change from baseline to Final Visit).

Any adjudication-confirmed event (secondary CV endpoint)

There was no difference in the occurrence of and time to any adjudicated event between the linagliptin group and the glimepiride group (linagliptin: 17.1% of patients, glimepiride: 17.8%; HR 0.96; 95% CI 0.85, 1.09).

Secondary diabetes-related endpoints

The changes from baseline to Final Visit were generally comparable for linagliptin and glimepiride treatment groups (Table 7). Although some results showed statistically significant treatment differences, they were small and were not considered clinically relevant. The transitions in UACR categories from baseline to Final Visit were comparable between the treatment groups; at Final Visit, 58.4% of patients in the linagliptin group still had a normal UACR (<30 mg/g), 14.1% had microalbuminuria (≥30 to ≤300 mg/g), and 1.4% of patients had macroalbuminuria (>300 mg/g). In the glimepiride group, 57.7% of patients had a normal UACR at baseline and at Final Visit, 16.0% of patients had microalbuminuria, and 1.4% of patients had macroalbuminuria.

TABLE 7
Change of diabetes-related secondary endpoints from baseline to Final
Visit - TS_D; HbA_{1c} and FPG: all observed cases, other parameters:
observed cases including values after rescue medication

| | Linagliptin | | Glimepiride | | | | |
|---|---|---|---|---|---|---|---|
| | | Change from | | Change from | | | |
| | | baseline, | | baseline, | Comparison vs. glimepiride | | |
| | Baseline, | adjusted* | Baseline, | adjusted* | Adjusted | | |
| | mean (SE) | mean (SE) | mean (SE) | mean (SE) | mean (SE) | 95% CI | p-value |
| HbA_{1c} [%] | 7.15 (0.01) | 0.06 (0.02) | 7.16 (0.01) | 0.15 (0.02) | −0.09 (0.03) | (−0.15, −0.03) | 0.0023 |
| FPG [mg/dL] | 140.0 (0.6) | 12.4 (0.9) | 139.8 (0.6) | 19.7 (0.9) | −7.3 (1.1) | (−9.7, −4.8) | <0.0001 |
| Total cholesterol | 176.5 (0.8) | −5.4 (0.7) | 176.6 (0.9) | −5.0 (0.7) | −0.4 (1.0) | (−2.4, 1.6) | 0.6823 |
| [mg/dL] | | | | | | | |
| LDL [mg/dL] | 95.3 (0.7) | −6.1 (0.6) | 94.8 (0.7) | −6.5 (0.6) | 0.4 (0.9) | (−1.3, 2.1) | 0.6400 |
| HDL [mg/dL] | 48.1 (0.2) | 0.7 (0.2) | 48.6 (0.3) | 0.3 (0.2) | 0.5 (0.2) | (0.0, 1.0) | 0.0497 |
| Triglycerides | 169.9 (2.6) | 1.7 (2.2) | 169.2 (3.0) | 5.2 (2.2) | −3.5 (3.1) | (−9.6, 2.7) | 0.2678 |
| [mg/dL] | | | | | | | |
| Creatinine | 0.95 (0.01) | 0.08 (0.01) | 0.94 (0.00) | 0.09 (0.01) | −0.01 (0.01) | (−0.03, 0.01) | 0.5165 |
| [mg/dL] | | | | | | | |
| eGFR (MDRD) | 76.5 (0.4) | −4.0 (0.3) | 77.1 (0.4) | −5.0 (0.3) | 1.0 (0.4) | (0.2, 1.8) | 0.0124 |
| [mL/min/1.73²] | | | | | | | |
| Urinary albumin | 76.9 (6.5) | 43.4 (5.8) | 58.5 (4.1) | 45.2 (5.9) | −1.9 (8.3) | (−18.1, 14.4) | 0.8231 |
| [mg/L] | | | | | | | |

*Adjusted for baseline

Conclusions:

This trial demonstrated non-inferiority of linagliptin to glimepiride for time to first occurrence of cardiovascular death, non-fatal myocardial infarction, or non-fatal stroke (3-point MACE) in patients with type 2 diabetes at elevated cardiovascular risk and on standard-of-care treatment (predominantly on metformin background treatment). Superiority of linagliptin over glimepiride was not achieved for the 3-point MACE endpoint. Similarly, no treatment difference was observed for the time to first 4-point MACE (hospitalisation for unstable angina pectoris in addition to 3-point MACE components). There were no significant treatment difference for all-cause mortality, cardiovascular death, and heart failure outcomes. The linagliptin treatment effect was more sustained compared with glimepiride; sustainability responder rates were significantly higher for combined endpoints of patients on trial medication at trial end, with HbA1c≤57.0%, without rescue medication, without >2% weight gain, and with or without moderate/severe hypoglycaemic episodes. Results of further treatment sustainability endpoints underlined this observation.

A significant and consistent risk reduction for hypoglycaemia was observed in the linagliptin group compared with the glimepiride group across all hypoglycaemia analyses. Patients in the linagliptin group showed a modest decrease in weight with a sustained and significant difference over time compared with the glimepiride group. HbA1c control was similar in both treatment groups over time, aside from transient lower HbA1c levels in the glimepiride group within the first weeks of treatment. Similar proportion of patients used rescue medication by the end of the trial; the time to first use differed due to the initial lower HbA1c levels in the glimepiride group. Analyses of all-cause mortality showed comparable results in both groups.

The safety profile of linagliptin in this trial population was consistent with the known safety profile of the drug; no new safety signal for linagliptin has been identified.

Further on:

A non-significant estimate favoring linagliptin was observed for non-cardiovascular death (HR 0.82 (95% CI, 0.66, 1.03 [p=0.08]).

### Particular Embodiments

A) Linagliptin Cardiovascular Safety Trial

This cardiovascular safety study was a randomized study in 6033 patients with early type 2 diabetes and increased CV risk or established complications who were treated with linagliptin 5 mg (3023) or glimepiride 1-4 mg (3010) added to standard of care (including background therapy with metformin in 83% of patients) targeting regional standards for HbA1c and CV risk factors. The mean age for study population was 64 years and included 2030 (34%) patients ≥70 years of age. The study population included 2089 (35%) patients with cardiovascular disease and 1130 (19%) patients with renal impairment with an eGFR <60m1/min/1.73m2 at baseline. The mean HbA1c at baseline was 7.15%.

The study was designed to demonstrate non-inferiority for the primary cardiovascular endpoint which was a composite of the first occurrence of cardiovascular death or a non-fatal myocardial infarction (Ml) or a non-fatal stroke (3P-MACE).

After a median follow up of 6.25 years (median time on treatment 5.86 years), linagliptin did not increase the risk of major adverse cardiovascular events (Table 8) as compared to glimepiride. Results were consistent for patients treated with or without metformin.

TABLE 8
Major adverse cardiovascular events (MACE) and mortality by treatment
group in the cardiovascular safety study

| | Linagliptin 5 mg | | Glimepiride (1-4 mg) | | |
|---|---|---|---|---|---|
| | Number of | Incidence | Number of | Incidence | Hazard |
| | Subjects | Rate per | Subjects | Rate per | Ratio |
| | (%) | 1000 PY* | (%) | 1000 PY* | (95% CI) |
| Number of patients | 3023 | | 3010 | | |
| Primary CV | 356 (11.8) | 20.7 | 362 (12.0) | 21.2 | 0.98 (0.84, 1.14)** |
| composite | | | | | |
| (Cardiovascular | | | | | |
| death, non-fatal | | | | | |
| MI, non-fatal stroke) | | | | | |

*PY = patient years
**Test on non-inferiority to demonstrate that the upper bound of the 95% CI for the hazard ratio is less than 1.3

The composite of treatment sustainability, a key secondary endpoint, was defined as the proportion of patients on study treatment following initial titration period (16 weeks) that maintain glycaemic control (HbA1c≤7.0%) at final visit without need for additional antidiabetic drug therapy (rescue medication) without any moderate (symptomatic with glucose value ≤70mg/dL) or severe (requiring assistance) hypoglycaemic episodes and without >2% weight gain. A higher number of patients on linagliptin (481, 16.0%) achieved this key secondary endpoint compared to glimepiride (305, 10.2%).

For the entire treatment period (median time on treatment 5.9 years) the rate of patients with moderate or severe hypoglycaemia was 6.5% on linagliptin versus 30.9% on glimepiride, severe hypoglycaemia occurred in 0.3% of patients on linagliptin versus 2.2% on glimepiride.

Accordingly:

A1. Linagliptin, optionally in combination with one or more other active agents, for use in the treatment of a diabetic (preferably type 2 diabetes) patient, wherein treatment of said patient with linagliptin does not increase the risk of three point major adverse cardiovascular events (3P-MACE) compared to a patient treated with glimepiride, wherein the three point major adverse cardiovascular events (3P-MACE) include cardiovascular death, nonfatal myocardial infarction (Ml) and/or nonfatal stroke.

A2. Linagliptin, optionally in combination with one or more other active agents, for use according to embodiment A1, wherein the risk is as shown in Table 1 of the description, such as e.g. characterized by the following hazard ratio (HR):

Hazard Ratio
(95% CI)
0.98 (0.84, 1.14)

A3. Linagliptin, optionally in combination with one or more other active agents, for use in the treatment of diabetic (preferably type 2 diabetes) patients, wherein treatment of said patients with linagliptin result in a significantly higher number of patients achieving sustained glycemic control (HbA1c<7%) without moderate or severe hypoglycemia and/or substantial weight gain >2% from baseline compared to patients treated with glimepiride, without a need for additional antidiabetic drug therapy.

A4. Linagliptin, optionally in combination with one or more other active agents, for use according to embodiment A3, wherein the treatment sustainability is as shown in Table 3 of the description, such as e.g. characterized by the following odds ratio:

Odds Ratio
(95% CI)
1.68 (1.43, 1.96)

A5. Linagliptin, optionally in combination with one or more other active agents, for use according to any one of embodiments A1 to A4 in the treatment of a diabetic (preferably type 2 diabetes) patient, wherein linagliptin effects the treatment as follows:

i) without increasing the risk of three point major adverse cardiovascular events (3P-MACE) compared to patients treated with glimepiride, wherein the three point major adverse cardiovascular events (3P-MACE) include cardiovascular death, nonfatal myocardial infarction (Ml) and/or nonfatal stroke,

ii) with resulting in a significantly higher number of patients achieving sustained glycemic control (HbA1c <7%) without moderate or severe hypoglycemia and/or substantial weight gain >2% from baseline compared to patients treated with glimepiride, without a need for additional antidiabetic drug therapy.

A6. Linagliptin, optionally in combination with one or more other active agents, for use according to any one of embodiments A1 to A5, wherein the patient is exposed to treatment for at least 5.86 years, and/or followed for at least 6.25 years.

A7. Linagliptin, optionally in combination with one or more other active agents, for use according to any one of embodiments A1 to A6, wherein the diabetic patient is at increased or high cardiovascular risk.

A8. Linagliptin, optionally in combination with one or more other active agents, for use according to any one of embodiments A1 to A7, wherein the diabetic patient has an increased or high risk of CV events, such as based on a pre-existing CV disease (e.g. selected from: myocardial infarction, coronary artery disease, percutaneous coronary intervention, coronary artery by-pass grafting, ischemic or hemorrhagic stroke, congestive heart failure, peripheral occlusive arterial disease), a vascular-related end-organ damage (e.g. selected from: impaired renal function, (micro- or macro)albuminuria, retinopathy), age >=70 years, and/or two or more CV risk factors (e.g. selected from: hypertension, smoking, dyslipidemia, duration of T2DM >10 years).

A9. Linagliptin, optionally in combination with one or more other active agents, for use according to any one of embodiments A1 to A8, wherein the diabetic patient has one or more of the following A), B), C) and D):

A) previous or existing vascular disease, such as selected from myocardial infarction, coronary artery disease, percutaneous coronary intervention, coronary artery by-pass grafting, ischemic or hemorrhagic stroke, congestive heart failure, and peripheral occlusive arterial disease,

B) vascular related diabetes end-organ damage, such as selected from (moderately) impaired renal function, (micro- or macro)albuminuria and retinopathy,

C) elderly age (e.g. >/=70 years), and

D) at least two cardiovascular risk factors selected from
advanced type 2 diabetes mellitus (e.g. >10 years duration),
hypertension,
current daily cigarette smoking,
dyslipidemia.

A10. Linagliptin, optionally in combination with one or more other active agents, for use according to any one of embodiments A1 to A9, wherein the patient has type 2 diabetes mellitus and insufficient glycaemic control, namely either treatment naïve or despite mono- or dual therapy with metformin and/or an alpha-glucosidase inhibitor or despite a sulphonylurea/glinide in mono- or dual therapy with metformin or an alpha-glucosidase inhibitor.

A11. Linagliptin, optionally in combination with one or more other active agents, for use according to any one of embodiments A1 to A10, wherein the treatment of said patient with linagliptin is monotherapy or as add-on therapy.

A12. Linagliptin, optionally in combination with one or more other active agents, for use according to any one of embodiments A1 to A11, wherein the treatment further comprises a step of identifying the diabetic patient at cardiovascular risk (e.g. having CV risk factors), especially identifying the diabetic patient at increased or high risk of cardiovascular events, prior to treatment with linagliptin.

A13. Linagliptin, optionally in combination with one or more other active agents, for use according to embodiment A12, wherein the risk is based on pre-existing CV disease (e.g. selected from: myocardial infarction, coronary artery disease, percutaneous coronary intervention, coronary artery by-pass grafting, ischemic or hemorrhagic stroke, congestive heart failure, peripheral occlusive arterial disease), a diabetes-related vascular end-organ damage (e.g. selected from: impaired renal function, (micro- or macro)albuminuria, retinopathy), age >=70 years, and/or two or more CV risk factors (e.g. selected from: hypertension, smoking, dyslipidemia, duration of T2DM >10 years), e.g. such as defined in embodiment A9.

### Further Aspects or Embodiments

B) Linagliptin Cardiovascular (Safety) and Renal Microvascular Outcome Trial

This cardiovascular and renal microvascular safety/outcome study was a randomized study in 6979 patients with type 2 diabetes with increased or high or even very high CV risk evidenced by a history of established macrovascular/cardiovscular or renal disease who were treated with linagliptin 5 mg (3494) or placebo (3485) added to standard of care targeting regional standards for HbA1c, CV risk factors and renal disease. The study population included 1,211 (17.4%) patients ≥75 years of age and 4,348 (62.3%) patients with renal impairment. Approximately 19% of the population had eGFR ≥45 to <60 mL/min/1.73 m2, 28% of the population had eGFR ≥30 to <45 mL/min/1.73 m2 and 15% had eGFR <30 mL/min/1.73 m2. The mean HbA1c at baseline was 8.0%.

The study was designed to demonstrate non-inferiority for the primary cardiovascular endpoint which was a composite of the first occurrence of cardiovascular death or a non-fatal myocardial infarction (Ml) or a non-fatal stroke (3P-MACE). The renal composite endpoint was defined as renal death or sustained end stage renal disease or sustained decrease of 40% or more in eGFR.

After a median follow up of 2.2 years (median time on treatment 1.9 years), linagliptin, when added to standard of care, did not increase the risk of major adverse cardiovascular events or renal outcome events (Table 9 and FIG. 2). There was no increased risk in hospitalization for heart failure which was an additional adjudicated endpoint observed compared to standard of care without linagliptin in patients with type 2 diabetes (Table 10).

TABLE 9
Major adverse cardiovascular events (MACE) and renal outcome events by treatment
group in the cardiovascular safety and renal microvascular outcome study

| | Linagliptin 5 mg | | Placebo | | |
|---|---|---|---|---|---|
| | Number of | Incidence | Number of | Incidence | Hazard |
| | Subjects | Rate per | Subjects | Rate per | Ratio |
| | (%) | 1000 PY* | (%) | 1000 PY* | (95% CI) |
| Number of | 3494 | | 3485 | | |
| patients | | | | | |
| Primary CV | 434 (12.4) | 57.7 | 420 (12.1) | 56.3 | 1.02 (0.89, 1.17)** |
| composite | | | | | |
| (Cardiovascular | | | | | |
| death, non-fatal | | | | | |
| MI, non-fatal | | | | | |
| stroke) | | | | | |
| Secondary renal | 327 (9.4) | 48.9 | 306 (8.8) | 46.6 | 1.04 (0.89, 1.22) |
| composite (renal | | | | | |
| death, ESRD, | | | | | |
| 40% sustained | | | | | |
| decrease in | | | | | |
| eGFR) | | | | | |

* PY = patient years
** Test on non-inferiority to demonstrate that the upper bound of the 95% CI for the hazard ratio is less than 1.3

TABLE 10
Hospitalization for heart failure and mortality by treatment group in the
cardiovascular safety and renal microvascular outcome study

| | Linagliptin 5 mg | | Placebo | | |
|---|---|---|---|---|---|
| | Number of | Incidence | Number of | Incidence | Hazard |
| | Subjects | Rate per | Subjects | Rate per | Ratio |
| | (%) | 1000 PY* | (%) | 1000 PY* | (95% CI) |
| Number of | 3494 | | 3485 | | |
| patients | | | | | |
| All-cause | 367 (10.5) | 46.9 | 373 (10.7) | 48.0 | 0.98 |
| mortality | | | | | (0.84, |
| | | | | | 1.13) |
| CV death | 255 (7.3) | 32.6 | 264 (7.6) | 34 | 0.96 |
| | | | | | (0.81, |
| | | | | | 1.14) |
| Hospitalization | 209 (6.0) | 27.7 | 226 (6.5) | 30.4 | 0.90 |
| for heart failure | | | | | (0.74, |
| | | | | | 1.08) |

* PY = patient years

In analyses for albuminuria progression (change from normoalbuminuria to micro- or macroalbuminuria, or from microalbuminuria to macroalbuminuria) the estimated hazard ratio was 0.86 (95% CI 0.78, 0.95) for linagliptin versus placebo. The microvascular endpoint was defined as the composite of renal death, sustained ESRD, sustained decrease of 50% in eGFR, albuminuria progression, use of retinal photocoagulation or intravitreal injections of an anti-VEGF therapy for diabetic retinopathy or vitreous haemorrhage or diabetes-related-blindness. The estimated hazard ratio for time to first occurrence for the composite microvascular endpoint was 0.86 (95% CI 0.78, 0.95) for linagliptin versus placebo, mainly driven by albuminuria progression.

Accordingly:

B1. Linagliptin, optionally in combination with one or more other active agents, for use according to any one of the preceding (particular) embodiments (e.g. A1 to A13) in the treatment of a diabetic (preferably type 2 diabetes) patient, wherein treatment of said patient with linagliptin does not increase the risk of three point major adverse cardiovascular events (3P-MACE) compared to a patient treated with placebo, wherein the three point major adverse cardiovascular events (3P-MACE) include cardiovascular death, nonfatal myocardial infarction (Ml) and/or nonfatal stroke.

B2. Linagliptin, optionally in combination with one or more other active agents, for use according to embodiment B1, wherein the risk is as characterized by the following hazard ratio (HR):

Hazard Ratio
(95% CI)
1.02 (0.89, 1.17)

B3. Linagliptin, optionally in combination with one or more other active agents, for use according to any one of the preceding embodiments in the treatment of a diabetic (preferably type 2 diabetes) patient, wherein treatment of said patient with linagliptin does not increase the risk of hospitalization for heart failure compared to a patient treated with placebo.

B4. Linagliptin, optionally in combination with one or more other active agents, for use according to embodiment B3, wherein the risk is as characterized by hazard ratio (HR) 0.90 (95% CI; 0.74, 1.08).

B5. Linagliptin, optionally in combination with one or more other active agents, for use according to any one of the preceding (particular) embodiments in the treatment of a diabetic (preferably type 2 diabetes) patient, wherein treatment of said patient with linagliptin does not increase the risk of key renal outcome events compared to a patient treated with placebo, wherein the key renal outcome events include renal death, sustained end stage renal disease (ESRD) and/or sustained decrease of 40% or more in estimated glomerular filtration rate (eGFR).

B6. Linagliptin, optionally in combination with one or more other active agents, for use according to embodiment B5, wherein the risk is as characterized by the following hazard ratio (HR):

Hazard Ratio
(95% CI)
1.04 (0.89, 1.22)

B7. Linagliptin, optionally in combination with one or more other active agents, for use according to any one of the preceding (particular) embodiments in the treatment of a diabetic (preferably type 2 diabetes) patient, wherein treatment of said patient with linagliptin prevents, delays the occurrence of, or reduces the risk of albuminuria progression compared to a patient treated with placebo, wherein the albuminuria progression includes change from normoalbuminuria to micro- or macroalbuminuria and/or change from microalbuminuria to macroalbuminuria.

B8. Linagliptin, optionally in combination with one or more other active agents, for use according to any one of the preceding (particular) embodiments in the treatment of a diabetic (preferably type 2 diabetes) patient, wherein treatment of said patient with linagliptin prevents, delays the occurrence of, or reduces the risk of microvascular renal and/or eye complications compared to a patient treated with placebo, wherein the microvascular renal and/or eye complications include renal death, sustained ESRD, sustained decrease of ≥50% in eGFR, albuminuria progression, use of retinal photocoagulation, use of intravitreal injections of an anti-VEGF therapy for diabetic retinopathy, vitreous hemorrhage and/or diabetes-related-blindness.

B9. Linagliptin, optionally in combination with one or more other active agents, for use according to any one of embodiments B1 to B8 in the treatment of a diabetic (preferably type 2 diabetes) patient, wherein linagliptin effects the treatment as follows:

i) without increasing the risk of three point major adverse cardiovascular events (3P-MACE), wherein the three point major adverse cardiovascular events (3P-MACE) include cardiovascular death, nonfatal myocardial infarction (Ml) and/or nonfatal stroke,

ii) without increasing the risk of hospitalization for heart failure,

iii) without increasing the risk of key renal outcome events, wherein the key renal outcome events include renal death, sustained end stage renal disease (ESRD) and/or sustained decrease of 40% or more in estimated glomerular filtration rate (eGFR),

iv) with preventing, delaying the occurrence or reducing the risk of albuminuria progression, wherein the albuminuria progression includes change from normoalbuminuria to micro- or macroalbuminuria and/or change from microalbuminuria to macroalbuminuria, and/or

v) with preventing, delaying the occurrence or reducing the risk of microvascular renal and/or eye complications, wherein the microvascular renal and/or eye complications include renal death, sustained ESRD, sustained decrease of 50% in eGFR, albuminuria progression, use of retinal photocoagulation, use of intravitreal injections of an anti-VEGF therapy for diabetic retinopathy, vitreous hemorrhage and/or diabetes-related-blindness.

B10. Linagliptin, optionally in combination with one or more other active agents, for use according to any one of embodiments B1 to B9, wherein the patient is exposed to treatment for at least 1.8 years or at least 1.9 years, and/or followed for at least 2.2 years.

B11. Linagliptin, optionally in combination with one or more other active agents, for use according to any one of embodiments B1 to B10, wherein the diabetic patient is at high or (very) increased vascular risk, such as at high or very high (vascular) risk for heart and kidney disease, especially at high or very high risk of cardiovascular and/or renal complications or events.

B12. Linagliptin, optionally in combination with one or more other active agents, for use according to any one of embodiments B1 to B11, wherein the patient has high or very high risk for heart and kidney disease such as a high risk of cardiovascular and/or renal events such as based on established macrovascular disease and/or renal disease (e.g. albuminuria and/or impaired renal function); such as e.g. wherein the diabetic patient has evidence of prevalent kidney disease or compromised kidney function, with or without macrovascular (cardiovascular) disease, such as defined by i) albuminuria and previous macrovascular disease and/or ii) impaired renal function with predefined urine albumin creatinine ratio (UACR).

B13. Linagliptin, optionally in combination with one or more other active agents, for use according to any one of embodiments B1 to B12, wherein the diabetic patient has:

(i) albuminuria (micro or macro) (such as e.g. urine albumin creatinine ratio (UACR) ≥30 mg/g creatinine or ≥30 mg/l (milligram albumin per liter of urine) or ≥30 μg/min (microgram albumin per minute) or ≥30 mg/24 h (milligram albumin per 24 hours)) and previous macrovascular disease, such as e.g. defined as one or more of a) to f):
a) previous myocardial infarction,
b) advanced coronary artery disease,
c) high-risk single-vessel coronary artery disease,
d) previous ischemic or haemorrhagic stroke,
e) presence of carotid artery disease,
f) presence of peripheral artery disease;

and/or

(ii) impaired renal function (e.g. with or without CV co-morbidities), such as e.g. defined by:
impaired renal function (e.g. as defined by MDRD formula) with an estimated glomerular filtration rate (eGFR) 15-45 mL/min/1.73 m² with any urine albumin creatinine ratio (UACR), or
impaired renal function (e.g. as defined by MDRD formula) with an estimated glomerular filtration rate (eGFR) ≥45-75 mL/min/1.73 m² with an urine albumin creatinine ratio (UACR) >200 mg/g creatinine or >200 mg/I (milligram albumin per liter of urine) or >200 pg/min (microgram albumin per minute) or >200 mg/24 h (milligram albumin per 24 hours).

B14. Linagliptin, optionally in combination with one or more other active agents, for use according to any one of embodiments B1 to B13, wherein the patient has type 2 diabetes mellitus and insufficient glycaemic control, namely either treatment naïve or pre-treated with any antidiabetic background therapy (including e.g. metformin, a sulfonylurea, a glinide, an insulin, an alpha-glucosidase inhibitor and/or a thiazolidinedione).

B15. Linagliptin, optionally in combination with one or more other active agents, for use according to any one of embodiments B1 to B14, wherein the treatment of said patient with linagliptin is monotherapy or as add-on therapy.

B16. Linagliptin, optionally in combination with one or more other active agents, for use according to any one of embodiments B1 to B15, wherein the treatment further comprises a step of identifying the diabetic patient at vascular risk, especially identifying the diabetic patient at high risk of cardiovascular and/or renal events, prior to treatment with linagliptin.

B17. Linagliptin, optionally in combination with one or more other active agents, for use according to any one of embodiments B1 to B16, wherein the treatment further comprises a step of identifying the diabetic patient at risk of heart failure, prior to treatment with linagliptin.

B18. Linagliptin, optionally in combination with one or more other active agents, for use according to embodiment B16 or B17, wherein the risk is based on established macrovascular disease and/or renal disease (e.g. albuminuria and/or impaired renal function), e.g. such as defined by i) albuminuria and previous macrovascular disease and/or ii) impaired renal function with predefined urine albumin creatinine ratio (UACR), e.g. such as defined in embodiment B13.

B19. Linagliptin, optionally in combination with one or more other active agents, for use according to any one of embodiments B1 to B18, wherein the patient has albuminuria, such as microalbuminuria (UACR 30-300 mg/g) or macroalbuminuria (UACR >300 mg/g),

and/or

impaired renal function, such as mild (eGFR ≥60 to <90 mL/min/1.73 m2), moderate (eGFR ≥45 to <60 mL/min/1.73 m2), moderate/severe (eGFR ≥30 to <45 mL/min/1.73 m2) or severe (eGFR <30 mL/min/1.73 m2) renal impairment.

Accordingly, long-term (cardiovascular and renal) safety profile of linagliptin across a broad range of type 2 diabetes patients has been established in two unique cardiovascular outcome trials A (Cardiovascular (Safety) and Renal Microvascular Outcome Trial) and B (Cardiovascular Safety Trial), including

A) patients at increased CV risk (early type 2 diabetes, ≥5 years duration, HbA1c 6.5-8%, cf. above aspect A), such as e.g. according to any one of embodiments A1 to A11; and

B) patients at high risk for CV or heart disease and/or chronic kidney disease (CKD) (established CV and/or kidney disease, HbA1c 6.5-10%, cf. above aspect B), such as e.g. according to any one of embodiments B1 to B19.

The present invention further provides linagliptin, optionally in combination with one or more other active agents, for use in the treatment of type 2 diabetes (especially characterized by cardiovascular and renal safety, especially over long-term duration of treatment), including in at-risk patients (e.g. as described herein, especially according to above aspects A and/or B) such as e.g. having or being at risk of atherosclerotic CV disease, heart failure and/or chronic kidney disease.

The present invention further provides linagliptin, optionally in combination with one or more other active agents, for use in the treatment of albuminuria (especially characterized by cardiovascular and renal safety, especially over long-term duration of treatment) in type 2 diabetes patients, including in at-risk patients (e.g. as described herein, especially according to above aspects A and/or B) such as e.g. having or being at risk of atherosclerotic CV disease, heart failure and/or chronic kidney disease.

1. A method for treating type 2 diabetes, comprising administering a pharmaceutically effective amount of linagliptin, optionally in combination with one or more other active agents, to a patient in need thereof wherein treatment with linagliptin does not increase the risk of at least one three point major adverse cardiovascular event (3P-MACE) compared to a patient treated with glimepiride, wherein the three point major adverse cardiovascular event is selected from the group consisting of cardiovascular death, nonfatal myocardial infarction (MI) and nonfatal stroke.

2. The method of claim 1, wherein the risk is characterized by the following hazard ratio:
Hazard Ratio
(95% CI)
0.98 (0.84, 1.14).

3. A method for treating type 2 diabetes, comprising administering a pharmaceutically effective amount of linagliptin, optionally in combination with one or more other active agents, to a patient in need thereof, wherein treatment with linagliptin results in result in sustained glycemic control (HbA1c<7%) without moderate or severe hypoglycemia and/or substantial weight gain >2% from baseline compared to a patient treated with glimepiride, without a need for additional antidiabetic drug rescue therapy.

4. The method according to claim 3, wherein the treatment sustainability is characterized by the following odds ratio:
Odds Ratio
(95% CI)
1.68 (1.43, 1.96).

5. A method for treating type 2 diabetes, comprising administering a pharmaceutically effective amount of linagliptin, optionally in combination with one or more other active agents, to a patient in need thereof, wherein treatment with linagliptin does not increase risk of deaths from all cause compared to a patient treated with glimepiride.

6. The method according to claim 5, wherein the risk is characterized by the following hazard ratio:
Hazard Ratio
(95% CI)
0.91 (0.78, 1.06).

7. The method according to claim 1, wherein the treatment with linagliptin:
ii) results in sustained glycemic control (HbA1c <7%) without moderate or severe hypoglycemia and/or substantial weight gain >2% from baseline compared to a patient treated with glimepiride, without a need for additional antidiabetic drug rescue therapy, and/or
iii) does not increase the risk of death from all cause compared to a patient treated with glimepiride,
iv) the risk of.

8. The method according to claim 1, wherein the patient is exposed to treatment for at least 5.86 years, and/or observed for at least 6.25 years.

9. The method according to claim 1, wherein the diabetic patient is at increased or high cardiovascular risk.

10. The method according to claim 1, wherein the diabetic patient has an increased or high risk of cardiovascular (CV) events, based on:
a pre-existing CV disease,
vascular-related end-organ damage
age >=70 years, and/or
two or more CV risk factors selected from from the group consisting of hypertension, smoking, dyslipidemia, and duration of type 2 diabetes mellitus >10 years.

11. The method according to claim 1, wherein the diabetic patient has one or more of the following A), B), C) and D):
A) previous or existing vascular disease selected from the group consisting of myocardial infarction, coronary artery disease, percutaneous coronary intervention, coronary artery by-pass grafting, ischemic or hemorrhagic stroke, congestive heart failure, and peripheral occlusive arterial disease,
B) vascular related diabetes end-organ damage selected from the group consisting of moderately impaired renal function, (micro- or macro)albuminuria, and retinopathy,
C) >/=70 years of age, and
D) at least two cardiovascular risk factors selected from
advanced type 2 diabetes mellitus >10 years duration,
hypertension,
current daily cigarette smoking, and
dyslipidemia.

12. The method according to claim 1, wherein the patient has insufficient glycaemic control and is treatment naive, or
the patient has insufficient glycaemic control (i) despite mono- or dual therapy with metformin and/or an alpha-glucosidase inhibitor or (ii) despite a sulphonylurea/glinide in mono- or dual therapy with metformin or an alpha-glucosidase inhibitor.

13. The method according to claim 1, wherein the treatment of said patient with linagliptin is monotherapy or as add-on therapy.

14. The method according to claim 1, wherein the treatment further comprises identifying the diabetic patient at increased or high risk of cardiovascular events, prior to treatment with linagliptin.

15. The method according to claim 14, wherein the risk is based on
pre-existing CV disease selected from the group consisting of myocardial infarction, coronary artery disease, percutaneous coronary intervention, coronary artery by-pass grafting, ischemic or hemorrhagic stroke, congestive heart failure, and peripheral occlusive arterial disease,
a diabetes-related vascular end-organ damage selected from the group consisting of impaired renal function, (micro- or macro)albuminuria, and retinopathy,
age >=70 years, and/or
two or more CV risk factors selected from the group consisting of hypertension, smoking, dyslipidemia, and duration of type 2 diabetes mellitus >10 years.

16-24. (canceled)

25. The method according to claim 1, wherein linagliptin is administered in an oral daily dose of 5 mg.

26. A method for treating type 2 diabetes, comprising administering a pharmaceutically effective amount of linagliptin, optionally in combination with one or more other active agents, to a patient in need thereof, wherein treatment with linagliptin does not increase the risk of at least one four point major adverse cardiovascular event (4P-MACE) compared to a patient treated with glimepiride, wherein the four point major adverse cardiovascular event is selected from the group consisting of cardiovascular death, nonfatal myocardial infarction (MI), nonfatal stroke, and hospitalization for unstable angina pectoris.
